(19) United States
(12) Patent Application Publication
(10) Pub. No.: US 2008/0139607 A1
(43) Pub. Date: Jun. 12, 2008

(54) New Compounds

(75) Inventors: Fredrik Almqvist, Umea (SE); Erik Chorell, Umea (SE); Pralay Das, West Bengal (IN); Hans Emtenas, Molndal (SE); Ola Fjellstrom, Molndal (SE); Mickael Mogemark, Molndal (SE); Magnus Polla, Molndal (SE); Veronica Aberg, Ostersund (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/930,232

(22) Filed: Oct. 31, 2007

Related U.S. Application Data
(60) Provisional application No. 60/863,836, filed on Nov. 1, 2006.

(51) Int. Cl. A61K 31/4365; C07D 471/04; C07D 211/76; A61K 31/4412; A61P 9/10; A61P 9/00; A61P 11/00; A61P 35/00

(52) U.S. Cl. 514/301; 546/114; 546/302; 514/345

(57) ABSTRACT

The present invention relates to new compounds of formula (I), (II), or (III)

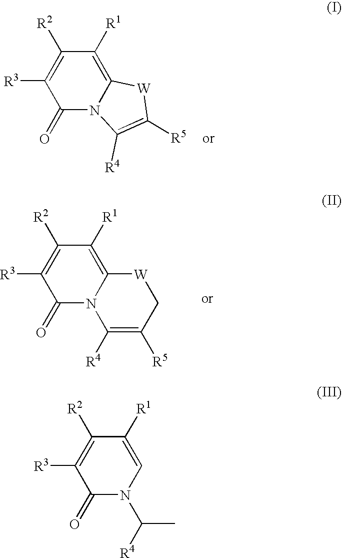

wherein R¹, R², R³, R⁴, R⁵ and W are as defined herein, and methods of using the compounds to inhibit PAI-1 and to treat PAI-1 related disorders.

### CROSS-REFERENCE TO RELATED APPLICATIONS

This application claims priority to U.S. provisional application Ser. No. 60/863,836 filed Nov. 1, 2006, which is incorporated herein by reference in its entirety.

### FIELD OF THE INVENTION

The present invention relates to new compounds of the formula (I), (II), or (III) and methods of using them to inhibit PAI-1 and to treat PAI-1 related disorders.

### BACKGROUND OF THE INVENTION

The serine protease PAI-1 (plasminogen activator inhibitor type 1) is one of the primary inhibitors of the fibrinolytic system. Fibrinolysis is the result of a series of enzymatic reactions resulting in the degradation of fibrin by plasmin. The activation of plasminogen is the central process in fibrinolysis. The cleavage of plasminogen to produce plasmin is accomplished by the plasminogen activators, tissue-type plasminogen activator (t-PA) or urokinase-type plasminogen activator (u-PA). The fibrinolytic system is not only responsible for the removal of fibrin from circulation but is also involved in several other biological processes including ovulation, embryogenesis, intima proliferation, angiogenesis, tumorigenesis, and atherosclerosis.

Elevated levels of PAI-1, due to increased production or activity, have been seen associated with a variety of diseases and conditions including those associated with impairment of fibrinolysis. These diseases and conditions include, but are not limited to, thrombosis, coronary heart disease, renal fibrosis, atherosclerotic plaque formation, pulmonary disease, myocardial ischemia, atrial fibrillation, coagulation syndromes, thromboembolic complications of surgery, peripheral arterial occlusion and pulmonary fibrosis. Other disorders include, but are not limited to, cancer, polycystic ovary syndrome, diabetes, and obesity.

For example, elevated levels of PAI-1 have been implicated in thrombotic diseases, e.g., diseases characterized by formation of a thrombus that obstructs vascular blood flow locally that detaches and embolizes to occlude blood flow downstream (Krishnamurti, Blood, 69,798 (1987); Reilly, Arteriosclerosis and Thrombosis, II, 1276, (1991); Carmeliet, Journal of Clinical Investigation, 92, 2756 (1993); Rocha, Fibrinolysis, 8, 294, 1994; Aznar, Haemostasis, 24, 243 (1994)). A Fab-fragment of a PAI-1 inhibiting antibody enhances fibrinolysis impaired in rats given endotoxin, leading to decreased tissue fibrin deposition (Abrahamsson, Thrombosis and Haemostasis, 75, 118 (1996).

Elevated PAI-1 levels have also been implicated in diseases such as polycystic ovary syndrome (Nordt, Journal of Clinical Endocrinology and Metabolism, 85, 4, 1563 (2000)), bone loss due to estrogen deficiency (Daci, Journal of Bone and Mineral Research, 15, 8, 1510 (2000)), cystic fibrosis, idiopathic pulmonary fibrosis, diabetes, chronic peridontitis, lymphomas, diseases associated with extracellular matrix accumulation, malignancies, diseases associated with neoangiogenesis, inflammatory diseases, vascular damage associated with infections, and diseases associated with increased levels such as breast and ovarian cancer.

The compounds of the invention are inhibitors of PAI-1 either as such or, in the case of prodrugs, after administration. The compounds of the invention are thus expected to be useful in PAI-1 related disorders, such as in the treatment or prophylaxis of thrombosis and hypercoagulability in blood and tissues of mammals, including man.

It is known that hypercoagulability may lead to thrombo-embolic diseases. Conditions associated with hypercoagulability and thrombo-embolic diseases which may be mentioned include protein C resistance and inherited or acquired deficiencies in antithrombin III, protein C, protein S and heparin cofactor II. Other conditions known to be associated with hyper-coagulability and thrombo-embolic disease include circulatory and septic shock, circulating antiphospholipid antibodies, homocysteinaemia, heparin induced thrombocytopenia and defects in fibrinolysis. The compounds of the invention are thus indicated both in the therapeutic and/or prophylactic treatment of conditions mentioned in this application.

Particular disease states which may be treated according to the present invention include, but are not limited to, venous thrombosis and pulmonary embolism, arterial thrombosis (e.g. in myocardial infarction, unstable angina, ischemic stroke and peripheral arterial thrombosis) and systemic embolism usually from the atrium during atrial fibrillation or from the left ventricle after transmural myocardial infarction.

Further indications include the therapeutic and/or prophylactic treatment of disseminated intravascular coagulation caused by bacteria, multiple trauma, intoxication or any other mechanism, fibrinolytic treatment when blood is in contact with foreign surfaces in the body, such as vascular grafts, vascular stents, vascular catheters, mechanical and biological prosthetic valves or any other medical device, and fibrinolytic treatment when blood is in contact with medical devices outside the body, such as during cardiovascular surgery using a heart-lung machine or in haemodialysis.

The compounds of the invention may also be combined and/or coadministered with any antithrombotic agent with a different mechanism of action, such as the antiplatelet agents acetylsalicylic acid, ticlopidine, clopidogrel, thromboxane receptor and/or synthetase inhibitors, fibrinogen receptor antagonists, prostacyclin mimetics, phosphodiesterase inhibitors, ADP-receptor (P₂T) antagonists, carboxypeptidase U inhibitors and thrombin inhibitors.

The compounds of the invention may further be combined and/or coadministered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction and stroke.

WO 01/36426, in the name of Washington University, discloses pyridones in treating or preventing Gram-negative bacterial infections.

Almqvist et al., J. Org. Chem. 2007, 72, 4917-4924 discloses diverse functionalization of thiazolo ring-fused 2-pyridones.

JP2005320346, WO 2005030716 and WO 200174793 all disclose PAI-1 inhibitors.

### SUMMARY OF THE INVENTION

The present invention provides compounds of formula (I), (II), or (III):

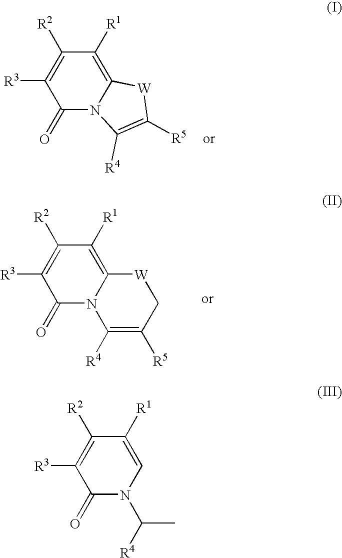

or a pharmaceutically acceptable salt or enantiomer thereof wherein: W is selected from S, SO, SO₂, O, P, PO, PO₂, and CH₂; R¹ is (CH₂)ₘD wherein m is a natural number being 0, 1, 2, 3, 4, or 5 and D is selected from hydrogen, alkyl, alkenyl, alkynyl, unsubstituted or substituted aryl, unsubstituted or substituted heteroaryl, substituted alkyl, substituted alkenyl, substituted alkynyl, and unsubstituted or substituted cycloalkyl; R² is selected from C₂-C₄-alkyl; unsubstituted or substituted isopentyl; unsubstituted or substituted C₆-C₁₀-alkyl; unsubstituted or substituted cycloalkylmethyl; unsubstituted or substituted (CH₂)ₘ-cycloalkyl, unsubstituted or substituted (CH₂)ₘ-aryl, wherein m is a natural number being 2, 3, 4, or 5; and (CH₂)ₙA wherein n is a natural number being 0, 1, 2, 3, 4, or 5 and A is selected from alkenyl, alkynyl, aryloxy, heteroaryl, substituted alkenyl, substituted alkynyl, substituted aryloxy, and substituted heteroaryl; R³ is selected from hydrogen, halogen, nitro, hydroxyalkyl, carboxy, and —NHR⁰ wherein R⁰ is selected from hydrogen, alkylsulfonyl, acyl, acyl substituted by acylamido and hydroxyalkyl; R⁴ is selected from CO₂Y, B(OY)₂, CHO, CH₂OY, CH(CO₂Y)₂, PO(OY)₂ wherein Y is selected from hydrogen, alkyl, alkenyl, alkynyl, aryl, heteroaryl, substituted alkyl, substituted alkenyl, substituted alkynyl, cycloalkyl, substituted aryl or substituted heteroaryl; tetrazolyl; and CONHZ wherein Z is selected from hydrogen, hydroxy, alkyl, alkylsulfonyl, arylsulfonyl, and cyanoalkyl; and R⁵ is selected from hydrogen, alkyl, alkoxy, unsubstituted or substituted aryl, and unsubstituted or substituted heteroaryl.

In some embodiments, for a compound of formula (I) or (III), W can be S or SO₂; R¹ can be (CH₂)ₘD wherein m can be 0 and D can be selected from unsubstituted or substituted cycloalkyl, unsubstituted or substituted aryl, and unsubstituted or substituted heteroaryl; R² can be selected from C₂-C₄-alkyl; isopentyl; C₆-C₁₀-alkyl; (CH₂)ₘ-aryl wherein m can be a natural number being 2, 3, 4, or 5; and (CH₂)ₙA wherein n can be a natural number being 0, 1, 2, 3, 4, or 5, and A can be substituted aryloxy; R³ can be selected from hydrogen, halogen, nitro, hydroxyalkyl, carboxy, and —NHR⁰ wherein R⁰ can be selected from hydrogen, alkylsulfonyl, acyl, acyl substituted by acylamido and hydroxyalkyl; R⁴ can be selected from CO₂Y wherein Y can be selected from hydrogen or alkyl; tetrazolyl; and CONHZ wherein Z can be selected from hydrogen, hydroxy, alkyl, alkylsulfonyl, arylsulfonyl, and cyanoalkyl; and R⁵ can be selected from hydrogen, alkyl, alkoxy, unsubstituted or substituted aryl, and unsubstituted or substituted heteroaryl.

In any of the above embodiments, R⁵ can be selected from hydrogen, alkyl, alkoxy, and unsubstituted or substituted aryl.

In any of the above embodiments, for a compound of formula (I) or (III), W can be S; R¹ can be (CH₂)ₘD wherein m can be 0 and D can be selected from cycloalkyl, unsubstituted or substituted aryl; R² can be selected from C₂-C₄-alkyl; isopentyl; C₆-C₁₀-alkyl; and (CH₂)ₙA wherein n can be 3 and A can be 2,4-dichlorophenoxy; R³ is selected from hydrogen, halogen, nitro, hydroxyalkyl, carboxy, —NHR⁰ wherein R⁰ can be selected from hydrogen, alkylsulfonyl, acyl, acyl substituted by acylamido and hydroxyalkyl; and R⁴ can be selected from CO₂Y wherein Y can be selected from hydrogen; tetrazolyl; and CONHZ wherein Z can be selected from alkylsulfonyl and arylsulfonyl; and R⁵ can be selected from hydrogen, methyl, methoxy, and phenyl.

In any of the above embodiments, aryl can be C₆₋₁₅ aryl, aryloxy can be C₆₋₁₅ aryloxy, alkenyl can be C₁₋₁₅ alkenyl, alkynyl can be C₁₋₁₅ alkynyl, cycloalkyl can be C₃₋₆ alkyl, and heteroaryl can be C₅₋₁₅ heteroaryl.

In any of the above embodiments, substituted aryl can be aryl substituted by one or more fluoro.

In any of the above embodiments, substituted aryl can be aryl substituted by one or more trifluoromethyl.

In any of the above embodiments, for a compound of formula (I), (II), or (III), the stereochemical configuration around the carbon which is covalently bound to R⁴ can be (R).

In any of the above embodiments, for a compound of formula (I), (II), or (III), the stereochemical configuration around the carbon which is covalently bound to R⁴ can be (S).

In any of the above embodiments, the compound can be selected from: (3R)-8-(3,4-Difluoro-phenyl)-7-heptyl-5-oxo-2,3-dihydro-5H-thiazolo[3,2-a]pyridine-3-carboxylic acid; (3R)—N-[8-(3,4-Difluoro-phenyl)-7-heptyl-5-oxo-2,3-dihydro-5H-thiazolo[3,2-a]pyridine-3-carbonyl]-methanesulfonamide; (2S)-2-[5-(3,4-Difluoro-phenyl)-4-heptyl-2-oxo-2H-pyridin-1-yl]-propionic acid; (3S)-8-(3,4-Difluoro-phenyl)-7-heptyl-5-oxo-2,3-dihydro-5H-thiazolo[3,2-a]pyridine-3-carboxylic acid amide; (3R)-8-(3,4-Difluoro-phenyl)-7-heptyl-3-(1H-tetrazol-5-yl)-2,3-dihydro-thiazolo[3,2-a]pyridin-5-one; (3R)-8-(3,4-Difluoro-phenyl)-7-heptyl-5-oxo-2,3-dihydro-5H-thiazolo[3,2-a]pyridine-3,6-dicarboxylic acid 3-methyl ester; (3R)-7-Heptyl-5-oxo-8-(3-trifluoromethyl-phenyl)-2,3-dihydro-5H-thiazolo[3,2-a]pyridine-3-carboxylic acid; (3R)—N-[7-Heptyl-5-oxo-8-(3-trifluoromethyl-phenyl)-2,3-dihydro-5H-thiazolo[3,2-a]pyridine-3-carbonyl]-benzenesulfonamide; (3R)-7-Butyl-5-oxo-8-(3-trifluoromethyl-phenyl)-2,3-dihydro-5H-thiazolo[3,2-a]pyridine-3-carboxylic acid; (3R)-7-Heptyl-6-nitro-5-oxo-8-(3-trifluoromethyl-phenyl)-2,3-dihydro-5H-thiazolo[3,2-a]pyridine-3-carboxylic acid; (3S)-8-(3,4-Difluoro-phenyl)-7-heptyl-5-oxo-2,3-dihydro-5H-thiazolo[3,2-a]pyridine-3-carboxylic acid; (3R)-7-Hexyl-6-nitro-5-oxo-8-(3-trifluoromethyl-phenyl)-2,3-dihydro-5H-thiazolo[3,2-a]pyridine-3-carboxylic acid; (3R)-7-(3-methylbutyl)-6-nitro-5-oxo-8-(3-trifluoromethyl-phenyl)-2,3-dihydro-5H-thiazolo[3,2-a]pyridine-3-carboxylic acid; (3R)-6-Amino-7-hexyl-5-oxo-8-(3-trifluoromethyl-phenyl)-2,3-dihydro-5H-thiazolo[3,2-a]pyridine-3-carboxylic acid; (3R)-7-Butyl-6-nitro-5-oxo-8-(3-trifluoromethyl-phenyl)-2,3-dihydro-5H-thiazolo[3,2-a]pyridine-3-carboxylic acid; (3R)-6-Amino-7-(3-methyl-butyl)-5-oxo-8-(3-trifluoromethyl-phenyl)-2,3-dihydro-5H-thiazolo[3,2-a]pyridine-3-carboxylic acid; (3R)-8-(3,4-Difluoro-phenyl)-7-heptyl-6-nitro-5-oxo-2,3-dihydro-5H-thiazolo[3,2-a]pyridine-3-carboxylic acid; (3R)-7-octyl-5-oxo-8-[3-(trifluoromethyl)phenyl]-2,3-dihydro-5H-[1,3]thiazolo[3,2-a]pyridine-3-carboxylic acid; (3R)-7-heptyl-5-oxo-8-(2-thienyl)-2,3-dihydro-5H-[1,3]thiazolo[3,2-a]pyridine-3-carboxylic acid; (3R)-7-heptyl-8-(1H-indol-3-yl)-5-oxo-2,3-dihydro-5H-[1,3]thiazolo[3,2-a]pyridine-3-carboxylic acid; (3R)-7-[3-(2,4-dichloro-phenoxy)-propyl]-5-oxo-8-phenyl-2,3-dihydro-5H-thiazolo[3,2-a]pyridine-3-carboxylic acid; (2S,3R)-8-(3,4-difluorophenyl)-7-heptyl-2-methoxy-5-oxo-2,3-dihydro-5H-[1,3]thiazolo[3,2-a]pyridine-3-carboxylic acid; 8-(3,4-difluorophenyl)-7-heptyl-5-oxo-5H-[1,3]thiazolo[3,2-a]pyridine-3-carboxylic acid; (2R,3R)-8-(3,4-difluorophenyl)-7-heptyl-5-oxo-2-phenyl-2,3-dihydro-5H-[1,3]thiazolo[3,2-a]pyridine-3-carboxylic acid; and (2R,3R)-8-(3,4-difluorophenyl)-7-heptyl-2-methyl-5-oxo-2,3-dihydro-5H-[1,3]thiazolo[3,2-a]pyridine-3-carboxylic acid.

The present invention also provides processes for the preparation of a compound according to any of the above embodiments comprising reacting a compound of formula (I) with Raney® nickel to give a compound of formula (III):

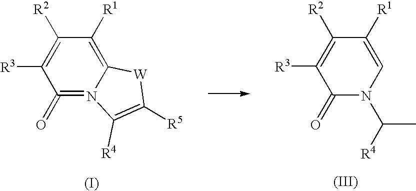

wherein R¹, R², R³, R⁴, R⁵, and W are as defined as above.

The present invention also provides pharmaceutical formulations comprising a compound according to any of the above embodiments in admixture with a pharmaceutically acceptable adjuvant, diluent, and/or carrier.

The present invention also provides methods for treating a disorder wherein inhibition of PAI-1 may be beneficial, which disorder is selected from thrombosis, coronary heart disease, renal fibrosis, atherosclerotic plaque formation, pulmonary disease, myocardial ischemia, atrial fibrillation, coagulation syndromes, thromboembolic complications of surgery, peripheral arterial occlusion, pulmonary fibrosis, cancer, polycystic ovary syndrome, diabetes, and obesity, by administering a compound according to any of the above embodiments to a mammal. In some embodiments, the compound is combined and/or coadministered with another antithrombotic agent.

### DESCRIPTION OF EMBODIMENTS

One object of the present invention is a compound of the formula (I), (II) or (III):

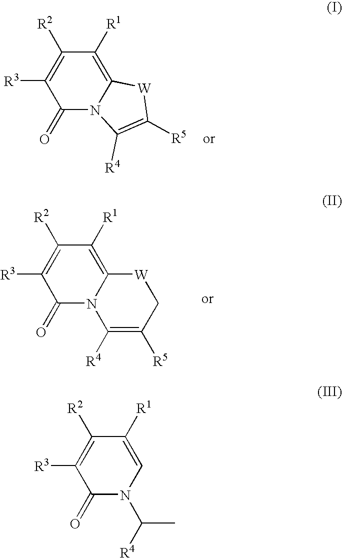

and pharmaceutically acceptable salts and enantiomers thereof wherein:

W is selected from S, SO, SO₂, O, P, PO, PO₂, and CH₂;

R¹ is (CH₂)ₘD wherein m is a natural number being 0, 1, 2, 3, 4, or 5 and D is selected from hydrogen, alkyl, alkenyl, alkynyl, unsubstituted or substituted aryl, unsubstituted or substituted heteroaryl, substituted alkyl, substituted alkenyl, substituted alkynyl, and unsubstituted or substituted cycloalkyl;

R² is selected from C₂-C₄-alkyl; unsubstituted or substituted isopentyl; unsubstituted or substituted C₆-C₁₀-alkyl; unsubstituted or substituted cycloalkylmethyl; unsubstituted or substituted (CH₂)ₘ-cycloalkyl, unsubstituted or substituted (CH₂)ₘ-aryl, wherein m is a natural number being 2, 3, 4, or 5; and (CH₂)ₙA wherein n is a natural number being 0, 1, 2, 3, 4, or 5 and A is selected from alkenyl, alkynyl, aryloxy, heteroaryl, substituted alkenyl, substituted alkynyl, substituted aryloxy, and substituted heteroaryl;

R³ is selected from hydrogen, halogen, nitro, hydroxyalkyl, carboxy, and —NHR⁰ wherein R⁰ is selected from hydrogen, alkylsulfonyl, acyl, acyl substituted by acylamido and hydroxyalkyl;

R⁴ is selected from CO₂Y, B(OY)₂, CHO, CH₂OY, CH(CO₂Y)₂, and PO(OY)₂ wherein Y is selected from hydrogen, alkyl, alkenyl, alkynyl, aryl, heteroaryl, substituted alkyl, substituted alkenyl, substituted alkynyl, cycloalkyl, substituted aryl or substituted heteroaryl; tetrazolyl; and CONHZ wherein Z is selected from hydrogen, hydroxy, alkyl, alkylsulfonyl, arylsulfonyl, and cyanoalkyl; and

R⁵ is selected from hydrogen, alkyl, alkoxy, unsubstituted or substituted aryl, and unsubstituted or substituted heteroaryl.

For Formulas (I) and (II), it should be noted that the bond between the carbon atom bonded to R⁴ and the carbon atom bonded to R⁵ may either be a single bond or a double bond.

### DEFINITIONS

The alkyl groups described herein, either alone or with the various substituents defined herein are preferably lower alkyl containing from one to four carbon atoms in the principal chain and up to 10 carbon atoms. They may be substituted, straight, or branched chain and include methyl, ethyl, propyl, isopropyl, butyl, hexyl, heptyl, octyl, nonyl, decyl and the like.

The alkoxy groups described herein, either alone or with the various substituents defined herein are preferably lower alkoxy containing from one to four carbon atoms in the principal chain and up to 10 carbon atoms. They may be substituted, straight, or branched chain and include methoxy, ethoxy, propoxy, isopropoxy, butoxy, hexoxy, heptoxy, octoxy, nonoxy, decoxy and the like.

The alkenyl groups described herein, either alone or with the various substituents defined herein are preferably lower alkenyl containing from two to four carbon atoms in the principal chain and up to 10 carbon atoms. They may be substituted, straight, or branched chain and include ethenyl, propenyl, isopropenyl, butenyl, hexenyl, heptyl, heptenyl, octenyl, nonenyl, docenyl and the like.

The alkynyl groups described herein, either alone or with the various substituents defined herein are preferably lower alkynyl containing from two to four carbon atoms in the principal chain and up to 10 carbon atoms. They may be substituted, straight, or branched chain and include ethynyl, propynyl, butynyl, hexynyl, heptynyl, octynyl, nonynyl, decynyl and the like.

The cycloalkyl groups described herein, either alone or with the various substituents defined herein are preferably lower cycloalkyl containing from three to seven carbon atoms in the principal chain and up to 10 carbon atoms. They may be substituted and include cyclopropyl, cyclobutyl, cyclopentyl, cyclohexyl, cycloheptyl, cyclooctyl, cyclononyl, cyclodecyl and the like.

The acyl groups described herein, either alone or with the various substituents defined herein are preferably lower acyl containing from one to four carbon atoms in the principal chain and up to 10 carbon atoms. They may be substituted, straight, or branched chain and include formyl, acetyl, propionyl, isopropionyl, butyryl, hexanoyl, heptanoyl, octanoyl, nonanoyl, decanoyl and the like.

The aryl moieties described here, either alone or with various substituents, contain from 6 to 15 carbon atoms and include phenyl, 1-naphthalenyl and 2-naphthalenyl. Substituents include alkoxy, halogen, hydroxyl, alkyl, aryl, alkenyl, acyl, acyloxy, nitro, amino, amido, trifluoromethyl, etc.

The aryloxy moieties described here, either alone or with various substituents, contain from 6 to 15 carbon atoms and include phenoxy, 1-naphthalenoxy and 2-naphthalenoxy. Substituents include alkoxy, halogen, hydroxyl, alkyl, aryl, alkenyl, acyl, acyloxy, nitro, amino, amido, trifluoromethyl, etc.

The heteroaryl moieties described here, either alone or with various substituents, contain from 3 to 15 carbon atoms and include furans, thiophenes, indoles, furyl, pyridyl, thienyl, tryptophane and the like. Substituents include alkanoxy, halogen, hydroxyl, alkyl, aryl, alkenyl, acyl, acyloxy, nitro, amino, amido, trifluoromethyl, etc.

The substituents of the substituted alkyl, alkenyl, alkynyl, aryl, and heteroaryl groups and moieties described herein, may be hydroxy, halogen, alkyl, alkenyl, alkynyl, aryl, heteroaryl and/or may contain nitrogen, oxygen, sulfur, halogens and include, for example, lower alkoxy such as methoxy, ethoxy, butoxy, halogen such as chloro or fluoro, nitro, amino, keto, and trifluoromethyl.

As used herein, the term “halogen” denotes a fluoro, chloro, bromo, or iodo group. The term “perhalo” denotes a group having the highest possible number of halogen atoms bonded thereto.

As used herein, when two or more groups are used in connection with each other, it means that each group is substituted by the immediately preceding group. For instance, trifluoromethylphenyl means a phenyl group substituted by a trifluoromethyl group.

As used herein, the term “prevent” or “prevention” is given its ordinary meaning and thus means the avoidance or alleviation of the serious consequences of a disease or a side-effect by early detection.

As used herein, the term “mammal” means a human or an animal such as monkeys, primates, dogs, cats, horses, cows, etc.

As used herein, the term “PAI-1 related disorder or disease” refers to any disease or condition that is associated with increased or enhanced expression or activity of PAI-1 or increased or enhanced expression or activity of a gene encoding PAI-1. The term “PAI-1 related disorder or disease” also refers to any disease or condition wherein inhibition of PAI-1 is beneficial.

As used herein, the single enantiomers, racemic mixtures and unequal mixtures of two enantiomers are within the scope of the invention, where such isomers exist. It should be understood that all the diastereomeric forms possible (pure enantiomers, racemic mixtures and unequal mixtures of two or more diastereomers), tautomers, and atropisomers are within the scope of the invention.

As used herein, the term “pharmaceutically acceptable salts” includes acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo or by freeze-drying). Salts may also be prepared by exchanging a counter-ion of a compound of the invention in the form of a salt with another counter-ion using a suitable ion exchange resin.

Suitable acids are non-toxic and include, but are not limited to, hydrochloric acid, hydrobromic acid, hydroiodic acid, sulphuric acid, nitric acid, acetic acid, citric acid, ascorbic acid, lactic acid, malic acid, and tartaric acid. Suitable bases are non-toxic and include, but are not limited to, sodium hydroxide, potassium hydroxide, lithium hydroxide, ammonia, methylamine, dimethylamine, trimethylamine, and triethylamine.

In the context of the present specification, the term “treat” also includes “prophylaxis” unless there are specific indications to the contrary. The term “treat” within the context of the present invention further encompasses to administer an effective amount of a compound of the present invention, to mitigate either a pre-existing disease state, acute or chronic, or a recurring condition. This definition also encompasses prophylactic therapies for prevention of recurring condition and continued therapy for chronic disorders.

The compounds of the present invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the present invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of the present invention, inert, pharmaceutically acceptable carriers can be either solid or liquid. Solid form preparations include powders, tablets, dispersable granules, capsules, cachets, and suppositories.

A solid carrier can be one or more substances, which may also act as diluents, flavouring agents, solubilizers, lubricants, suspending agents, binders, or tablet disintegrating agents; it can also be an encapsulating material.

In powders, the carrier is a finely divided solid, which is in mixture with the finely divided compound of the present invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogenous mixture is then poured into conveniently sized moulds and allowed to cool and solidify.

Suitable carriers are magnesium carbonate, magnesium stearate, talc, lactose, sugar, pectin, dextrin, starch, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavouring agents, stabilizers, and thickening agents as desired. Aqueous solutions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will according to one embodiment of the present invention include 0.05% to 99% weight (percent by weight), according to an alternative embodiment from 0.10 to 50% weight, of the compound of the present invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

The above-mentioned subject-matter for a pharmaceutical composition comprising a compound according to the present invention is applied analogously for a pharmaceutical composition comprising a combination according to the present invention.

In another embodiment, the present invention relates to a compound of the formula (I) or (III) wherein:

W is selected from S and SO₂;

R¹ is (CH₂)ₘD wherein m is 0 and D is selected from unsubstituted or substituted cycloalkyl, unsubstituted or substituted aryl, and unsubstituted or substituted heteroaryl; R² is selected from C₂-C₄-alkyl; isopentyl; C₆-C₁₀-alkyl; (CH₂)ₘ-aryl, wherein m is a natural number being 2, 3, 4, or 5; or (CH₂)ₙA wherein n is a natural number being 0, 1, 2, 3, 4, or 5 and A is substituted aryloxy;

R³ is selected from hydrogen, halogen, nitro, hydroxyalkyl, carboxy, —NHR⁰ wherein R⁰ is selected from hydrogen, alkylsulfonyl, acyl, acyl substituted by acylamido and hydroxyalkyl;

R⁴ is selected from CO₂Y wherein Y is selected from hydrogen or alkyl; tetrazolyl; and CONHZ wherein Z is selected from hydrogen, hydroxy, alkyl, alkylsulfonyl, arylsulfonyl, and cyanoalkyl; and

R⁵ is selected from hydrogen, alkyl, alkoxy, unsubstituted or substituted aryl, unsubstituted or substituted heteroaryl.

In another embodiment R⁵ is selected from hydrogen, alkyl, alkoxy, and unsubstituted or substituted aryl.

In another embodiment, the present invention relates to a compound of the formula (I) or (III) wherein:

W is S;

R¹ is (CH₂)ₘD wherein m is 0 and D is selected from cycloalkyl, unsubstituted or substituted aryl;

R² is selected from C₂-C₄-alkyl; isopentyl; C₆-C₁₀-alkyl; and (CH₂)ₙA wherein n is 3 and A is 2,4-dichlorophenoxy;

R³ is selected from hydrogen, halogen, nitro, hydroxyalkyl, carboxy, —NHR⁰ wherein R⁰ is selected from hydrogen, alkylsulfonyl, acyl, acyl substituted by acylamido and hydroxyalkyl;

R⁴ is selected from CO₂Y wherein Y is selected from hydrogen; tetrazolyl; and CONHZ wherein Z is selected from alkylsulfonyl and arylsulfonyl; and

R⁵ is selected from hydrogen, methyl, methoxy, and phenyl.

In another embodiment aryl is C₆₋₁₅ aryl, aryloxy is C₆₋₁₅ aryloxy, alkenyl is C₁₋₁₅ alkenyl, alkynyl is C₁₋₁₅ alkynyl, cycloalkyl is C₃₋₆ alkyl, and heteroaryl is C₅₋₁₅ heteroaryl.

In another embodiment substituted aryl is aryl substituted by one or more fluoro.

In another embodiment substituted aryl is aryl substituted by one or more trifluoromethyl.

In another embodiment the stereochemical configuration around the carbon which is covalently bound to R⁴ is (R).

In another embodiment the stereochemical configuration around the carbon which is covalently bound to R₄ is (S).

Specific compounds are denoted in Examples 1-25.

Another object of the present invention is a process for the preparation of a compound as disclosed above comprising reacting a compound of formula (I) with Raney® nickel to give a compound of formula (III):

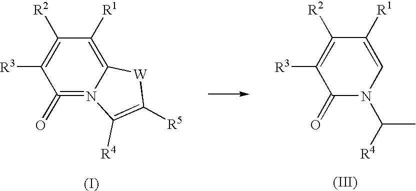

wherein R¹, R², R³, R⁴, R⁵, and W are as defined above.

Another object of the present invention is a compound as disclosed above for use in medicine.

Another object of the present invention is a pharmaceutical formulation comprising a compound as disclosed above in admixture with a pharmaceutically acceptable adjuvant, diluent and/or carrier.

According to another aspect of present invention, there is provided a compound as disclosed above for use in the treatment of a disorder wherein inhibition of PAI-1 may be beneficial, which disorder is selected from thrombosis, coronary heart disease, renal fibrosis, atherosclerotic plaque formation, pulmonary disease, myocardial ischemia, atrial fibrillation, a coagulation syndrome, a thromboembolic complication of surgery, peripheral arterial occlusion, pulmonary fibrosis, cancer, polycystic ovary syndrome, diabetes, and obesity.

Another object of the present invention is the use of a compound above, in the manufacture of a medicament for treating a disorder wherein inhibition of PAI-1 may be beneficial, which disorder is selected from thrombosis, coronary heart disease, renal fibrosis, atherosclerotic plaque formation, pulmonary disease, myocardial ischemia, atrial fibrillation, a coagulation syndrome, a thromboembolic complication of surgery, peripheral arterial occlusion, pulmonary fibrosis, cancer, polycystic ovary syndrome, diabetes, and obesity.

In another embodiment the compound is combined and/or coadministered with another antithrombotic agent.

Another object of the present invention is a method for treating a disorder wherein inhibition of PAI-1 may be beneficial, which disorder is selected from thrombosis, coronary heart disease, renal fibrosis, atherosclerotic plaque formation, pulmonary disease, myocardial ischemia, atrial fibrillation, a coagulation syndrome, a thromboembolic complication of surgery, peripheral arterial occlusion, pulmonary fibrosis, cancer, polycystic ovary syndrome, diabetes, and obesity, by administering to a mammal of a compound above.

In another embodiment the compound is combined and/or coadministered with another antithrombotic agent.

In the following, the present invention is illuminated by the following non-limiting Examples. It should be understood that these examples are for illustrative purposes only and are not to be construed as limiting the invention in any manner.

When used, the expressions “comprise” and “comprising” denote “include” and “including” but not limited to. Thus, other ingredients, carriers and additives may be present.

### EXAMPLES

### Abbreviations

rt or RT—room temperature

t—triplet

s—singlet

d—doublet

q—quartet

quint—quintet

m—multiplet

br—broad

bs—broad singlet

dm—doublet of multiplet

bt—broad triplet

dd—doublet of doublet

### General Experimental Procedures

General synthesis: All reactions were carried out under inert atmosphere, with dry solvents and anhydrous conditions, unless otherwise stated. MeCN, CH₂Cl₂ and 1,2-dichlorethane were distilled from calcium hydride and THF was distilled from potassium. DMF was distilled and dried over 3 Å molecular sieves. EtOH was dried over 3 Å molecular sieves. HCl (g) was passed through concentrated H₂SO₄ prior to use. All microwave reactions were carried out in a monomode reactor (Smith Synthesizer, Biotage AB) using Smith Process Vials™ sealed with Teflon septa and aluminum crimp tops. TLC was performed on Silica Gel 60 F₂₅₄ (Merck) using UV light detection. Flash column chromatography (eluents given in brackets) employed normal phase silica gel (Matrex, 60 Å, 35-70 μm, Grace Amicon). The ¹H and ¹³C NMR spectra were recorded at 298 K with a Bruker DRX-400 spectrometer in CDCl₃ {residual CHCl₃ (δ_{H} 7.26 ppm) or CDCl₃ (δ_{C} 77.0 ppm) as internal standard}, or MeOD-d₄ {residual CD₂HOD (δ_{H} 3.30 ppm) or CD₃OD (δ_{C} 49.0 ppm) as internal standard}, or DMSO-d₆ {residual DMSO (δ_{H} 2.49 ppm) or DMSO (δ_{C} 40.0 ppm) as internal standard}. Overlapping carbon signals were resolved by HSQC experiments on a Bruker DRX-500. IR spectra were recorded on an ATI Mattson Genesis Series FTIR™ spectrometer. Optical rotations were measured with a Perkin-Elmer 343 polarimeter at 20° C. HRMS data were recorded with fast atom bombardment (FAB+) ionization on a JEOL JMS-SX 102 spectrometer.

In the following, general procedures for preparation are disclosed, including the preparation of the following Examples. The correlation between the compounds and Examples is as follows:

| Example | Compound |
|---|---|
| 1 | 13 |
| 3 | 68 |
| 4 | 20 |
| 5 | 53 |
| 6 | 72 |
| 7 | 12 |
| 8 | 65 |
| 10 | 36 |
| 11 | 76 |
| 12 | 38 |
| 13 | 40 |
| 15 | 39 |
| 17 | 37 |

### 1. General Procedure for Imidate and Thiazoline Derivative Synthesis:

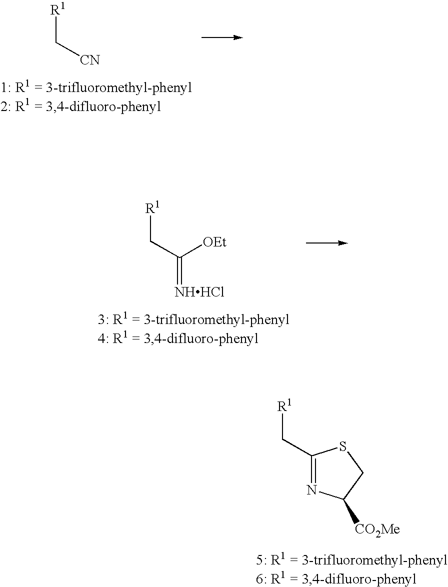

### (4R)-2-(3-Trifluoromethyl-benzyl)-4,5-dihydro-thiazole-4-carboxylic acid methyl ester (5)

Dry HCl(g) was passed through a solution of 3-(trifluoromethyl)phenylacetonitrile 1 (2 g, 10.8 mmol) in dry EtOH (1 ml) during 4 h at 0° C. After standing overnight at room temperature, the mixture was concentrated to give 3 as white solid, which were used in the next step without further purification. Triethylamine (1 ml, 7.85 mmol) was slowly added to a suspension of L-cysteinemethyl ester hydrochloride (1.34 g, 7.85 mmol) in dry CH₂Cl₂ (9 ml) at 0° C. After 20 min, a suspension of 3 (1.5 g, 5.61 mmol) in CH₂Cl₂ (2.5 ml) was added, and the mixture was allowed to attain room temperature overnight. Then, the mixture was diluted with CH₂Cl₂ and washed with water, saturated aqueous NaHCO₃, and brine. The aqueous layers were extracted with CH₂Cl₂ and the combined organic layers were dried (Na₂SO₄), filtered, and concentrated. The residue was purified by flash column chromatography (heptane/ethyl acetate, 3:7) to give A²-thiazoline (5) as a solid (1.43 g, 84%). ¹H NMR (400 MHz, CDCl₃) δ 7.55-7.41 (m, 4H), 5.12 (dd, J=8.65, 9.63 Hz, 1H), 3.93 (s, 2H), 3.81 (s, 3H), 3.60 (dd, J=8.79, 11.31 Hz, 1H), 3.51 (dd, J=9.42, 11.30 Hz, 1H); ¹³C NMR (100 MHz, CDCl₃) δ 172.61, 171.01, 136.46, 132.43, 130.94, 129.09, 125.80, 124.15, 77.85, 52.72, 40.29, 35.93.

Compound 6 was synthesized using the following method described for 5.

### 2. Meldrum's Acid Derivative Synthesis:

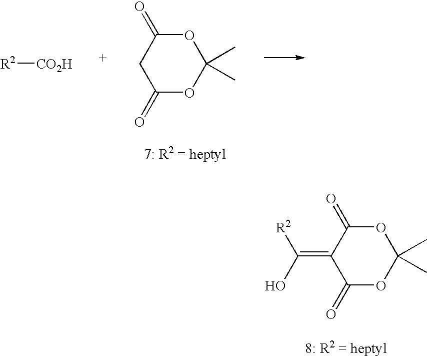

### 5-(1-Hydroxy-octylidene)-2,2-dimethyl-[1,3]dioxane-4,6-dione (8)

Oxalyl chloride (10 ml, 114 mmol) and DMF (0.1 ml) were added to a solution of octanoic acid (7) (5.85 g, 40.6 mmol) in dry CH₂Cl₂ (80 ml). After being stirred for 30 min at room temperature, the solution was refluxed for 1.5 h, cooled to room temperature, and concentrated. The residue was co-concentrated three times from dry CH₂Cl₂ and dissolved in dry CH₂Cl₂ (40 ml). The solution was added slowly during 1 h to a stirred solution of Meldrum's acid (5.19 g, 36 mmol) and DMAP (8.62 g, 70.3 mmol) in dry CH₂Cl₂ (80 ml) at −10° C. The resulting solution was allowed to attain room temperature and was then stirred for 3 h before being diluted with CH₂Cl₂ and washed with aqueous KHSO₄ (2%), water, and brine. The aqueous layers were extracted with CH₂Cl₂, and the combined organic layers were dried (Na₂SO₄), filtered, and concentrated. The residue was recrystallized from Et₂O to give 8 as oil (10.54 g, 96%). ¹H NMR (400 MHz, CDCl₃) δ 3.01 (t, J=7.55 Hz, 2H), 1.67 (s, 6H), 1.35-1.22 (m, 10H), 0.83 (t, J=7.21 Hz, 3H); ¹³C NMR (100 MHz, CDCl₃) δ 198.20, 170.48, 160.07, 104.64, 91.14, 35.62, 31.51, 29.21, 28.77, 26.68, 26.04, 22.46, 13.93.

3. Synthesis of 2-pyridones:

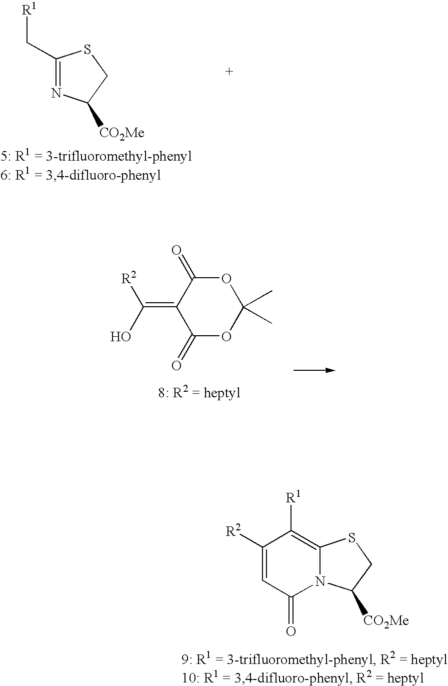

### (3R)-7-Heptyl-5-oxo-8-(3-trifluoromethyl-phenyl)-2,3-dihydro-5H-thiazolo[3,2-a]-pyridine-3-carboxylic acid methyl ester (9)

### Method 1.

Dry HCl(g) was passed through a solution of 5 (300 mg, 1.00 mmol) and 8 (600 mg, 2.22 mmol) in 1,2-dichloroethane (7 ml) during 15 min at 0° C. The solution was stirred for 11 h at 64° C. The mixture was cooled to room temperature, diluted with CH₂Cl₂, and washed with water, saturated aqueous NaHCO₃, and brine. The aqueous layers were extracted with CH₂Cl₂, and the combined organic layers were dried (Na₂SO₄), filtered, and concentrated. Purification by flash column chromatography heptane/ethyl acetate, 1:4 gave 2-pyridinone 9 as oil (400 mg, 89%).

### Method 2.

Trifluoroacetic acid (0.25 ml, 3.3 mmol) was added in a solution of 5 (500 mg, 1.65 mmol) and 8 (937 mg, 3.3 mmol) in 1,2-dichloroethane (10 ml). The vial was capped and heated with microwave irradiation (normal absorption) at 120° C. for 140 sec. The mixture was cooled to room temperature, diluted with CH₂Cl₂, and washed with water, saturated aqueous NaHCO₃, and brine. The aqueous layers were extracted with CH₂Cl₂, and the combined organic layers were dried (Na₂SO₄), filtered, and concentrated. Purification by flash column chromatography heptane/ethyl acetate, 1:4 gave 2-pyridinone 9 as oil (673 mg, 87.3%). ¹H NMR (400 MHz, CDCl₃) δ 7.64 (d, J=8.27 Hz, 1H), 7.57-7.53 (m, 1H), 7.44 (d, J=7.24 Hz, 1H), 6.25 (s, 1H), 5.65 (dd, J=8.27, 10.83 Hz, 1H), 3.84 (s, 3H), 3.67 (t, J=8.27 Hz, 1H), 3.46 (d, J=11.89 Hz, 1H), 2.21 (t, J=7.6 Hz, 2H), 1.37-1.32 (m, 2H), 1.25-1.12 (m, 8H), 0.82 (t, J=7.12 Hz, 3H); ¹³C NMR (100 MHz, CDCl₃) δ 168.42, 161.34, 155.79, 146.70, 137.36, 133.54, 129.35, 127.00, 125.00, 122.47, 114.81, 114.28, 63.52, 53.30, 33.11, 31.73, 31.44, 28.98, 28.94, 28.69, 22.46, 13.93.

### (3R)-8-(3,4-Difluoro-phenyl)-7-heptyl-5-oxo-2,3-dihydro-5H-thiazolo[3,2-a]pyridine-3-carboxylic acid methyl ester (10)

Dry HCl(g) was passed through a solution of 6 (730 mg, 2.68 mmol) and 8 (1.45 g, 5.36 mmol) in 1,2-dichloroethane (12 ml) during 15 min at 0° C. The solution was stirred for 11 h at 64° C. The mixture was cooled to room temperature, diluted with CH₂Cl₂, and washed with water, saturated aqueous NaHCO₃, and brine. The aqueous layers were extracted with CH₂Cl₂, and the combined organic layers were dried (Na₂SO₄), filtered, and concentrated. Purification by flash column chromatography heptane/ethyl acetate, 1:4 gave 2-pyridinone 10 as oil (980 mg, 87%). ¹³C NMR (100 MHz, CDCl₃) δ 168.29, 161.14, 155.68, 151.35, 148.74, 146.70, 133.14, 126.45, 119.10, 117.6, 114.07, 113.94, 63.40, 53.15, 32.93, 31.55, 31.35, 28.88, 28.72, 28.61, 22.37, 13.85.

### 4. Methyl Ester Hydrolysis:

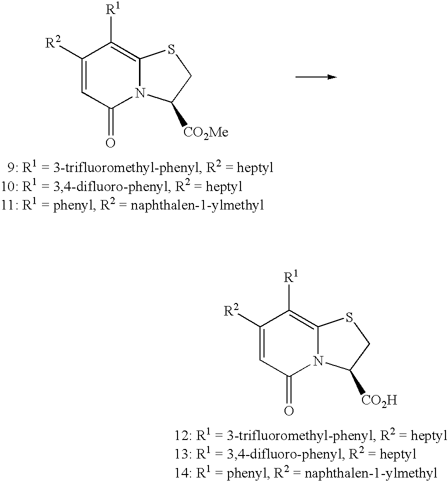

### General Procedure for Carboxylic Acid Synthesis:

### (3R)-7-Heptyl-5-oxo-8-(3-trifluoromethyl-phenyl)-2,3-dihydro-5H-thiazolo[3,2-a]pyridine-3-carboxylic acid (12)

1 ml MeOH was added to 9 (35 mg, 0.08 mmol) and allowed to stir for five minutes. Then 0.1 M LiOH (aq.) (0.75 ml) was added and the solution was stirred over-night. Then concentrated and co-concentrated three times with ethanol. The mixture was dissolved in 1 ml MeOH then added Amberlite® IR-120+(500 mg) and stirred well for 10 min. The solid phase was washed with MeOH. The filtrate was concentrated from CH₂Cl₂ gave 12 as a solid (33.4 mg, quant.) ¹³C NMR (100 MHz, CDCl₃) δ 168.82, 162.55, 157.41, 148.25, 136.88, 131.26, 133.39, 129.45, 126.82, 125.21, 122.39, 116.85, 113.74, 64.64, 33.16, 31.41, 31.37, 29.08, 29.02, 28.63, 22.44, 13.91.

### (3R)-8-(3,4-Difluoro-phenyl)-7-heptyl-5-oxo-2,3-dihydro-5H-thiazolo[3,2-a]pyridine-3-carboxylic acid (13)

7 ml MeOH was added to 10 (150 mg, 0.36 mmol) and allowed to stir for five minutes. Then 0.1 M LiOH (aq.) (3.4 ml) was added and the solution was stirred over-night. Then concentrated and co-concentrated three times with ethanol. The mixture was dissolved in 5 mL MeOH then added Amberlite® IR-120+(500 mg) and stirred well for 10 min. The solid phase was washed with MeOH. The filtrate was concentrated from CH₂Cl₂ gave 13 as a solid (146 mg, quant.). ¹H NMR (400 MHz, CDCl₃) δ 7.23-7.21 (m, 1H), 7.06-7.04 (m, 1H), 6.95 (s, 1H), 6.32 (s, 1H), 5.76-5.75 (m, 1H), 3.77-3.74 (m, 1H), 3.7-3.63 (m, 1H), 2.2-2.19 (m, 2H), 1.30-1.34 (m, 2H), 1.22-1.12 (m, 8H), 0.83 (t, J=7.2 Hz, 3H); ¹³C NMR (100 MHz, CDCl₃) δ 171.15, 162.49, 157.00, 151.47, 148.92, 133.07, 127.99, 126.45, 119.1, 117.84, 115.92, 113.50, 65.66, 33.13, 31.52, 29.20, 29.17, 28.75, 22.52, 13.97.

Compound 14 was synthesized using the following methods described for 12 and 13.

5. Amide Synthesis from Methyl Ester:

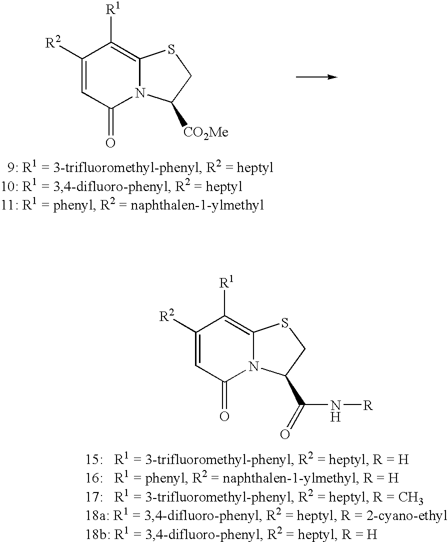

### General Procedure for Amide Synthesis:

### (3R)-7-Heptyl-5-oxo-8-(3-trifluoromethyl-phenyl)-2,3-dihydro-5H-thiazolo[3,2-a]pyridine-3-carboxylic acid amide (15)

9 (150 mg, 0.33 mmol) was dissolved in NH₃ (g) saturated MeOH (5 ml) at rt and the solution was then heated to 40° C. in a flask that was sealed with a rubber septum. Additional NH₃ (g) saturated MeOH was added portion wise (3×1 ml) to the reaction over a total of 29 hours to obtain full conversion into the amide. Concentration from CH₂Cl₂ gave 15 as a pale yellow foam (144.9 mg, quant.). ¹H NMR (400 MHz, CDCl₃) δ 7.84 (bs, 1H), 7.66 (d, J=8.00 Hz, 1H), 7.56-7.51 (m, 2H), 7.42 (d, J=7.60 Hz, 1H), 6.29 (s, 1H), 5.73 (d, J=7.19 Hz, 1H), 5.46 (bs, 1H), 4.00 (d, J=10.88 Hz, 1H), 3.53 (dd, J=8.25, 10.88 Hz, 1H), 2.22 (t, J=7.34 Hz, 2H), 1.36 (m, 2H), 1.22-1.12 (m, 8H), 0.83 (t, J=7.03 Hz, 3H); ¹³C NMR (100 MHz, CDCl₃) δ 168.89, 162.37, 156.62, 148.19, 137.24, 133.54, 129.62, 129.47, 127.00, 125.31, 116.20, 114.17, 64.56, 33.21, 31.58, 30.14, 29.21, 29.12, 28.81, 22.61, 14.09.

### (3R)-7-Naphthalen-1-ylmethyl-5-oxo-8-phenyl-2,3-dihydro-5H-thiazolo[3,2-a]pyridine-3-carboxylic acid amide (16)

Prepared as described for 15; Starting from 11 (300 mg, 0.70 mmol) gave 16 as a pale yellow foam (289 mg, quant.). [α]D-26 (c 0.5, MeOH); IR v/cm-1 3056, 2935, 1634, 1563, 1480, 1412; ¹H NMR (400 MHz, MeOD-d₄) δ 7.80 (dd, J=2.5, 5.7 Hz, 1H), 7.72 (d, J=8.2 Hz, 1H), 7.61 (dd, J=2.3, 7.0 Hz, 1H), 7.44-7.31 (m, 10H), 7.21 (d, J=7.0 Hz, 1H), 5.71 (s, 1H), 5.52 (dd, J=2.1, 8.9 Hz, 1H), 4.04-3.90 (m, 2H), 3.71 (dd, J=8.9, 11.8 Hz, 1H), 3.41 (dd, J=2.1, 11.8 Hz, 1H); ¹³C NMR (100 MHz, MeOD-d₄) δ 171.98, 163.49, 156.89, 150.57, 137.67, 135.37, 135.30, 133.02, 131.42, 131.01, 130.07 (2C), 129.72, 129.54, 128.93, 128.70, 127.18, 126.71, 126.49, 124.83, 118.34, 114.77, 66.06, 37.65, 32.99; HRMS (FAB+) calcd for [M+H]⁺C₂₅H₂₁N₂O₂S 413.1324, obsd 413.1327.

### (3R)-7-Heptyl-5-oxo-8-(3-trifluoromethyl-phenyl)-2,3-dihydro-5H-thiazolo[3,2-a]pyridine-3-carboxylic acid methylamide (17)

0.2 ml 2 (M)THF methyl amine was added in a solution of 9 (10 mg, 0.02 mmol) in 1 ml MeOH at rt. The mixture was heated at 50° C. for overnight. Solvent was evaporated and co-concentrated twice from methanol. Purification by silica gel chromatography (heptane:EtOAc, 3:7) gave 17 (6.8 mg, 76%). ¹H NMR (400 MHz, CDCl₃) δ 7.89 (bs, 1H), 7.66 (d, J=9.34 Hz, 1H), 7.55 (d, J=9.34 Hz, 1H), 7.51 (s, 1H), 6.28 (s, 1H), 5.70 (d, J=8.12 Hz, 1H), 4.07 (d, J=11.37 Hz, 1H), 3.55-3.50 (m, 1H), 2.83 (d, J=4.8 Hz, 3H), 2.21 (t, J=7.31 Hz, 2H), 1.36-1.11 (m, 10H), 0.83 (t, J=7.31 Hz, 3H); ¹³C NMR (100 MHz, CDCl₃) δ 167.42, 162.37, 156.44, 148.38, 144.29, 137.30, 133.57, 129.53, 127.05, 125.32, 116.27, 114.13, 64.72, 33.23, 31.60, 30.26, 29.25, 29.15, 28.84, 26.71, 22.64, 14.10.

### (3R)-8-(3,4-Difluoro-phenyl)-7-heptyl-5-oxo-2,3-dihydro-5H-thiazolo[3,2-a]pyridine-3-carboxylic acid (2-cyano-ethyl)-amide (18a)

10 (150 mg, 0.37 mmol), 1,3-dicyclohexylcarbodiimide (76.3 mg, 0.37 mmol) and 1-hydroxybenzotriazole (50 mg, 0.37 mmol) were dissolved in DMF (3 ml) and stirred at 0° C. then 3-aminopropionitrile was added drop wise to the above mixture. After stirring for 10 h at 0° C. then for 14 h at ambient temperature, the reaction mixture was poured into ice-cold H₂O and extracted with EtOAc. The combined organic extracts were washed successively with 1N HCl, H₂O, 8% NaHCO₃ solution, H₂O and brine. The organic layer was dried over Na₂SO₄ and concentrated under reduced pressure. Purification by silica gel chromatography (EtOAc, 100%) gave 18a (102.2 mg, 60.2%). ¹H NMR (400 MHz, CDCl₃) δ 8.41 (t, J=5.72 Hz, 1H), 7.27-7.14 (m, 1H), 7.07-7.02 (m, 1H), 6.96 (s, 1H), 6.29 (s, 1H), 5.71 (d, J=8.17 Hz, 1H), 3.99-3.95 (m, 1H), 3.60-3.46 (m, 3H), 2.65-2.61 (m, 2H), 2.23 (t, J=7.76 Hz, 2H), 1.36 (t, J=7.2 Hz, 2H), 1.25-1.14 (m, 8H), 0.86 (t, J=7.35 Hz, 3H); ¹³C NMR (100 MHz, CDCl₃) δ 167.57, 162.27, 157.15, 151.61, 148.85, 132.82, 126.43, 119.17, 117.90, 117.41, 116.24, 113.69, 113.62, 64.51, 35.89, 33.07, 31.50, 29.80, 29.04, 29.00, 28.74, 22.52, 17.99, 13.98.

### 8-(3,4-Difluoro-phenyl)-7-heptyl-5-oxo-2,3-dihydro-5H-thiazolo[3,2-a]pyridine-3-carboxylic acid amide (18b)

Compound 18b was prepared as described for 15.

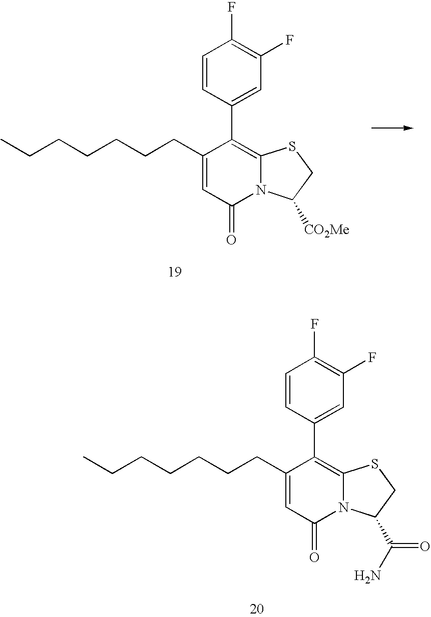

### (3S)-8-(3,4-Difluoro-phenyl)-7-heptyl-5-oxo-2,3-dihydro-5H-thiazolo[3,2-a]pyridine-3-carboxylic acid methyl ester (19)

Prepared as described for 10; Starting from thiazoline (synthesized from D-cysteine methyl ester hydrochloride using same method as (5) (200 mg, 0.74 mmol) gave 19 as oil (270 mg, 87%).

### (3S)-8-(3,4-Difluoro-phenyl)-7-heptyl-5-oxo-2,3-dihydro-5H-thiazolo[3,2-a]pyridine-3-carboxylic acid amide (20)

19 (40 mg, 0.09 mmol) was dissolved in NH₃ (g) saturated MeOH (3 ml) at rt and the solution was then heated to 40° C. in a flask that was sealed with a rubber septum. Additional NH₃ (g) saturated MeOH was added portion wise (3×1 ml) to the reaction over a total of 29 hours to obtain full conversion into the amide. Concentration from CH₂Cl₂ gave 20 as a pale yellow foam (38.6 mg, quant.). ¹H NMR (400 MHz, CDCl₃) δ 7.77 (bs, 1H), 7.20-7.21 (m, 1H), 7.04-7.06 (m, 1H), 6.96-6.94 (m, 1H), 6.25 (s, 1H), 5.86 (bs, 1H), 5.71 (d, 7.65 Hz, 1H), 3.96-3.92 (m, 1H), 3.55-3.51 (m, 1H), 2.22 (t, J=7.65 Hz, 2H), 1.35-1.14 (m, 10H), 0.84 (t, J=7.10 Hz), 3H); ¹³C NMR (100 MHz, CDCl₃) δ 169.27, 162.3, 156.70, 151.72, 149.23, 148.43, 133.25, 126.66, 119.39, 118.03, 115.52, 114.05, 64.73, 33.24, 31.69, 30.40, 29.86, 29.22, 28.94, 22.71, 14.18.

### 6. Nitration in R³ Position:

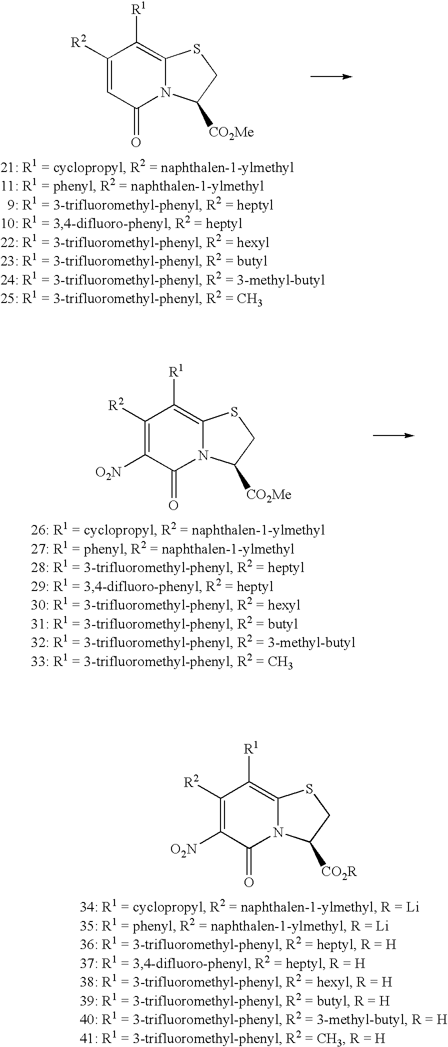

Compounds 22, 23, 24 and 25 were synthesized according to procedures described for 10.

### General Procedure for Nitration in R³ Position:

### (3R)-8-Cyclopropyl-7-naphthalen-1-ylmethyl-6-nitro-5-oxo-2,3-dihydro-5H-thiazolo[3,2-a]pyridine-3-carboxylic acid methyl ester (26)

To a mixture of 21 (750 mg, 1.92 mmol) and NaNO₂ (139 mg, 2.0 mmol) was added 60 ml CH₂Cl₂. A balloon filled with oxygen gas was connected to the flask via a rubber septum and 2.5 ml TFA was added dropwise at rt. After 5 h the brown solution was neutralized with NaHCO₃ (aq.) and then extracted with CH₂Cl₂. The organic phase was dried over Na₂SO₄ (s), filtrated and concentrated. Purification by silica gel chromatography (heptane:EtOAc, 1:1→1:2) gave 26 as a yellow foam (703 mg, 84%): [α]_{D}-324 (c 0.5, CHCl₃); IR v/cm⁻¹ 1753, 1657, 1589, 1525, 1485, 1437, 1371, 1346, 1207, 1167, 1030, 1010, 961, 797, 769, 734; ¹H NMR (400 MHz, CDCl₃) δ 8.04 (d, J=8.3 Hz, 1H), 7.85 (d, J=8.0 Hz, 1H), 7.72 (d, J=8.2 Hz, 1H), 7.58-7.47 (m, 2H), 7.37-7.31 (m, 1H), 7.04 (d, J=7.1 Hz, 1H), 5.75-5.70 (m, 1H), 4.69-4.50 (m, 2H), 3.82 (s, 3H), 3.75-3.66 (m, 1H), 3.52 (dd, J=2.0, 12.0 Hz, 1H), 1.23-1.13 (m, 1H), 0.66-0.48 (m, 4H); ¹³C NMR (100 MHz, CDCl₃) δ 167.51, 153.18, 152.24 (splitted), 148.30 (splitted), 139.51, 133.40, 131.99, 131.30, 128.66, 127.25, 126.21, 125.66, 125.31, 124.44, 122.52, 112.10, 63.37, 53.32, 31.46, 31.11, 11.11, 7.54, 7.10; HRMS (FAB+) calcd for [M+H]⁺C₂₃H₂₁N₂O₅S 437.1171, obsd 437.1180.

### (3R)-7-Naphthalen-1-ylmethyl-6-nitro-5-oxo-8-phenyl-2,3-dihydro-5H-thiazolo[3,2-a]pyridine-3-carboxylic acid methyl ester (27)

By following the procedure described for the preparation of 26 from 21, 11 (750 mg, 1.75 mmol), NaNO₂ (127 mg, 1.84 mmol), 50 ml CH₂Cl₂ and 2.0 ml TFA gave 27 as a yellow foam (737 mg, 89%) after purification with silica gel chromatography (heptane:EtOAc, 1:1→11:4). [α]_{D}-258 (c 0.5, CHCl₃); IR v/cm⁻¹ 1747, 1657, 1583, 1523, 1475, 1438, 1352, 1214, 1152, 1006, 780, 734, 700; ¹H NMR (400 MHz, CDCl₃) δ 7.77 (d, J=8.1 Hz, 1H), 7.69 (d, J=8.3 Hz, 1H), 7.55 (d, J=8.3 Hz, 1H), 7.43-7.31 (m, 3H), 7.16-7.09 (m, 3H), 7.09-7.02 (m, 1H), 7.00-6.96 (m, 1H), 6.90 (d, J=7.5 Hz, 1H), 5.80 (dd, J=2.4, 8.7 Hz, 1H), 4.30-4.12 (m, 2H), 3.90 (s, 3H), 3.75 (dd, J=8.8, 11.9 Hz, 1H), 3.54 (dd, J=2.4, 12.0 Hz, 1H); ¹³C NMR (100 MHz, CDCl₃) δ 167.48, 153.57, 151.55, 146.83, 139.43, 134.29, 133.43, 132.09, 131.29, 129.90, 129.34, 128.85, 128.80, 128.76, 128.58, 127.55, 126.07, 125.64, 125.58, 125.28, 122.54, 114.93, 64.43, 53.74, 31.91, 31.87; HRMS (FAB+) calcd for [M+H]⁺ C₂₆H₂₁N₂O₅S 473.1171, obsd 473.1180.

### (3R)-7-Heptyl-6-nitro-5-oxo-8-(3-trifluoromethyl-phenyl)-2,3-dihydro-5H-thiazolo[3,2-a]pyridine-3-carboxylic acid methyl ester (28)

To a mixture of 9 (149 mg, 0.33 mmol) and NaNO₂ (27 mg, 0.39 mmol) was added 12 ml CH₂Cl₂. A balloon filled with oxygen gas was connected to the flask via a rubber septum and 0.46 ml TFA was added dropwise at rt. After 5 h the brown solution was neutralized with NaHCO₃ (aq.) and then extracted with CH₂Cl₂. The organic phase was dried over Na₂SO₄ (s), filtrated and concentrated. Purification by silica gel chromatography (heptane:EtOAc, 1:1) gave 28 as a yellow foam (120 mg, 73%).

### (3R)-8-(3,4-Difluoro-phenyl)-7-heptyl-6-nitro-5-oxo-2,3-dihydro-5H-thiazolo[3,2-a]pyridine-3-carboxylic acid methyl ester (29)

To a mixture of 10 (213 mg, 0.51 mmol) and NaNO₂ (38.5 mg, 0.56 mmol) was added 12 ml CH₂Cl₂. A balloon filled with oxygen gas was connected to the flask via a rubber septum and 0.66 ml TFA was added dropwise at rt. After 5 h the brown solution was neutralized with NaHCO₃ (aq.) and then extracted with CH₂Cl₂. The organic phase was dried over Na₂SO₄ (s), filtrated and concentrated. Purification by silica gel chromatography (heptane:EtOAc, 1:1) gave 29 as a yellow foam (172 mg, 72%).

Compounds 30-33 were synthesized according to above mentioned methods.

General Procedure for Hydrolysis into Lithium Carboxylates 34 and 35

The methyl ester was dissolved in THF:MeOH (3:7) to a concentration of 50 mM then 1.0 eq. 0.1 M LiOH (aq.) was added dropwise at 0° C. The solution was allowed to attain rt while stirring overnight and was then concentrated and lyophilized from MeCN:H₂O (˜1:2) to give quantitative yields of the lithium carboxylates.

### (3R)-8-Cyclopropyl-7-naphthalen-1-ylmethyl-6-nitro-5-oxo-2,3-dihydro-5H-thiazolo[3,2-a]pyridine-3-lithium carboxylate (34)

[α]_{D}-216 (c 0.5, MeOH); IR v/cm⁻¹ 1632, 1614, 1485, 1386, 1333, 1217, 1168; ¹H NMR (400 MHz, MeOD-d₄) δ 8.14 (d, J=7.8 Hz, 1H), 7.88 (d, J=7.4 Hz, 1H), 7.75 (d, J=7.9 Hz, 1H), 7.62-7.47 (m, 2H), 7.38-7.30 (m, 1H), 7.08 (d, J=6.5 Hz, 1H), 5.55 (d, J=8.4 Hz, 1H), 4.75-4.50 (m, 2H), 3.88-3.78 (m, 1H), 3.67 (d, J=11.3 Hz, 1H), 1.35-1.20 (m, 1H), 0.68-0.51 (m, 4H); ¹³C NMR (100 MHz, MeOD-d₄) δ 173.10, 155.86, 155.52, 149.09, 140.77, 135.18, 134.07, 133.07, 129.86, 128.36, 127.43, 126.88, 126.50, 126.04, 124.00, 114.05, 68.15, 34.21, 32.37, 12.34, 8.40, 7.77.

### (3R)-7-Naphthalen-1-ylmethyl-6-nitro-5-oxo-8-phenyl-2,3-dihydro-5H-thiazolo[3,2-a]pyridine-3-lithium carboxylate (35)

[α]_{D}-83 (c 0.5, DMSO); IR v/cm⁻¹ 1744, 1649, 1477, 1342, 1220, 1155, 800, 778, 703; ¹H NMR (400 MHz, DMSO-d₆) δ 13.85 (bs, 1H), 7.88-7.83 (m, 1H), 7.78-7.72 (m, 2H), 7.49-7.37 (m, 3H), 7.26-7.06 (m, 6H), 5.74 (dd, J=1.9, 9.3 Hz, 1H), 4.26-4.05 (m, 2H), 3.95 (dd, J=9.3, 12.0 Hz, 1H), 3.62 (dd, J=1.9, 12.0 Hz, 1H); ¹³C NMR (100 MHz, DMSO-d₆) δ 169.14, 154.22, 153.21, 145.64, 138.79, 134.97, 133.40, 132.69, 131.23, 130.35, 130.07, 129.16, 129.09 (2C), 128.88, 127.64, 126.71, 126.25, 125.73, 125.44, 123.25, 113.84, 64.99, 32.39, 31.94.

### (3R)-7-Heptyl-6-nitro-5-oxo-8-(3-trifluoromethyl-phenyl)-2,3-dihydro-5H-thiazolo[3,2-a]pyridine-3-carboxylic acid (36)

The hydrolysis of 28 required a total of 1.5 eq. LiOH (0.1 M aq.) and the obtained lithium carboxylate 36 was therefore treated with Amberlite® IR120+ ion-exchange resin to yield the corresponding carboxylic acid of 36. Characterization was performed on the carboxylic acid. ¹H NMR (400 MHz, CDCl₃) δ 7.74 (d, J=7.6 Hz, 1H), 7.63 (t, J=7.4 Hz, 1H), 7.57 (s, 1H), 7.50 (d, J=7.2 Hz, 1H), 5.83 (d, J=7.08 Hz, 1H), 3.81-3.70 (m, 2H), 2.37-2.29 (m, 2H), 1.35-0.99 (m, 8H), 0.80 (t, J=7.2 Hz, 3H); ¹³C NMR (100 MHz, CDCl₃) δ 168.63, 161.4, 154.53, 152.45, 149.54, 138.18, 135.41, 133.67, 131.56, 129.91, 127.10, 126.10, 64.95, 31.68, 31.23, 29.72, 29.37, 29.01, 28.16, 22.39, 13.89.

### (3R)-8-(3,4-Difluoro-phenyl)-7-heptyl-6-nitro-5-oxo-2,3-dihydro-5H-thiazolo[3,2-a]pyridine-3-carboxylic acid (37)

The hydrolysis of 29 required a total of 1.5 eq. LiOH (0.1 M aq.) and the obtained lithium carboxylate 37 was therefore treated with Amberlite® IR120+ ion-exchange resin to yield the corresponding carboxylic acid of 47.

Compounds 38-41 were synthesized according to 36.

### 7. Nitro to Amine in R³ Position:

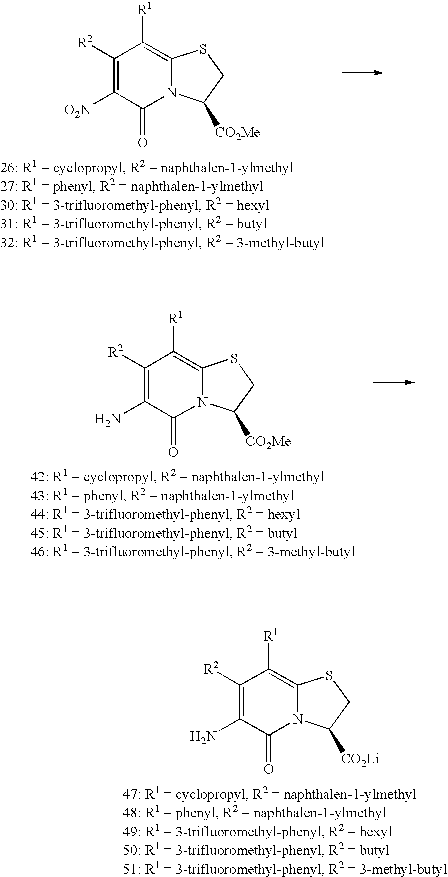

### General Procedure for the Nitro Reduction to Amine and Hydrolysis:

### (3R)-6-Amino-8-cyclopropyl-7-naphthalen-1-ylmethyl-5-oxo-2,3-dihydro-5H-thiazolo[3,2-a]pyridine-3-carboxylic acid methyl ester (42)

26 (685 mg, 1.57 mmol) was dissolved in 11 ml acetic acid and then activated Zn dust (472 mg, 7.22 mmol) was added in portions in order to control the temperature of the reaction. After 5 h of stirring at rt the solvent was removed under vacuum. The residue was dissolved in CH₂Cl₂, neutralized with Na₂CO₃ (aq.) and extracted with CH₂Cl₂. The organic phase was dried over Na₂SO₄ (s), filtrated and concentrated. Purification by silica gel chromatography (heptane:EtOAc, 1:1→1:9) gave 42 as a foam (571 mg, 90%): [α]_{D}-190 (c 0.5, CHCl₃); IR v/cm⁻¹ 1742, 1638, 1575, 1510, 1434, 1353, 1287, 1215, 1179, 1149, 1010, 960, 793, 772, 731; ¹H NMR (400 MHz, CDCl₃) δ 8.14 (d, J=8.4 Hz, 1H), 7.82 (d, J=8.0 Hz, 1H), 7.67 (d, J=8.2 Hz, 1H), 7.57-7.50 (m, 1H), 7.50-7.44 (m, 1H), 7.29-7.23 (m, 1H), 6.96 (d, J=7.0 Hz, 1H), 5.59 (dd, J=2.2, 8.2 Hz, 1H), 4.55-4.39 (m, 2H), 3.86 (bs, 2H), 3.76 (s, 3H), 3.57 (dd, J=8.4, 11.8 Hz, 1H), 3.42 (dd, J=2.3, 11.7 Hz, 1H), 1.52-1.44 (m, 1H), 0.66-0.58 (m, 2H), 0.54-0.45 (m, 2H); ¹³C NMR (100 MHz, CDCl₃) δ 168.35, 156.25, 133.41, 132.22, 131.90, 131.76, 131.70, 128.42, 127.55, 126.73, 125.75, 125.33, 125.27, 122.96, 122.72, 114.27, 62.69, 52.71, 31.29, 30.30, 11.36, 6.54, 6.51; HRMS (FAB+) calcd for [M]⁺ C₂₃H₂₂N₂O₃S 406.1351, obsd 406.1429.

### (3R)-6-Amino-7-naphthalen-1-ylmethyl-5-oxo-8-phenyl-2,3-dihydro-5H-thiazolo[3,2-a]pyridine-3-carboxylic acid methyl ester (43)

By following the procedure described for the preparation of 42 from 26, 27 (623 mg, 1.32 mmol) and Zn dust (404 mg, 6.18 mmol) in 9 ml acetic acid gave 43 (474 mg, 81%) after purification with silica gel chromatography (heptane:EtOAc, 1:1→1:2). [α]_{D}-167 (c 0.5, CHCl₃); IR v/cm⁻¹ 1740, 1633, 1572, 1488, 1440, 1315, 1207, 1155, 1073, 977, 906, 793, 731, 702; ¹H NMR (400 MHz, CDCl₃) δ 7.89-7.83 (m, 2H), 7.75 (d, J=8.2 Hz, 1H), 7.50-7.42 (m, 2H), 7.41-7.36 (m, 1H), 7.26-7.11 (m, 6H), 5.74 (dd, J=2.4, 8.2 Hz, 1H), 4.07 (s, 2H), 3.86 (s, 5H), 3.65 (dd, J=8.2, 11.7 Hz, 1H), 3.47 (dd, J=2.4, 11.8 Hz, 1H); ¹³C NMR (100 MHz, CDCl₃) δ 168.44, 156.51, 136.97, 133.63, 132.55, 131.98, 131.70, 131.08, 129.51 (broad), 128.91 (broad), 128.57, 128.39 (2C, broad), 127.77, 127.17, 125.90, 125.57, 125.50, 125.27, 123.66, 122.92, 117.52, 63.77, 53.09, 31.69, 31.52; HRMS (FAB+) calcd for [M]⁺ C₂₆H₂₂N₂O₃S 442.1351, obsd 442.1358.

Compounds 44-46 were synthesized according to 42.

### (3R)-6-Amino-8-cyclopropyl-7-naphthalen-1-ylmethyl-5-oxo-2,3-dihydro-5H-thiazolo[3,2-a]pyridine-3-lithium carboxylate (47)

Prepared as described for 34 and 35. [α]_{D}-179 (c 0.5, MeOH); IR v/cm⁻¹ 1612, 1557, 1505, 1396, 1274, 1024, 782; ¹H NMR (400 MHz, MeOD-d₄) δ 8.25 (d, J=8.5 Hz, 1H), 7.88 (d, J=8.1 Hz, 1H), 7.72 (d, J=8.2 Hz, 1H), 7.62-7.53 (m, 1H), 7.53-7.48 (m, 1H), 7.34-7.27 (m, 1H), 7.03 (dd, J=7.1, 0.7 Hz, 1H), 5.48 (dd, J=1.5, 8.3 Hz, 1H), 4.62-4.51 (m, 2H), 3.72 (dd, J=8.4, 11.3 Hz, 1H), 3.59 (dd, J=1.5, 11.3 Hz, 1H), 1.52-1.43 (m, 1H), 0.64-0.57 (m, 2H), 0.57-0.50 (m, 1H), 0.48-0.40 (m, 1H); ¹³C NMR (100 MHz, MeOD-d₄) δ 174.35, 158.30, 136.15, 135.42, 134.25, 133.67, 133.42, 131.24, 129.81, 128.02, 127.15, 126.70, 126.65, 124.78, 124.32, 116.42, 67.67, 34.02, 31.67, 12.75, 7.59, 7.36.

### (3R)-6-Amino-7-naphthalen-1-ylmethyl-5-oxo-8-phenyl-2,3-dihydro-5H-thiazolo[3,2-a]pyridine-3-lithium carboxylate (48)

Prepared as described for 34 and 35. [α]_{D}-165 (c 0.5, MeOH); IR v/cm⁻¹ 1621, 1566, 1487, 1440, 1395, 1372, 1315, 790, 766, 685; ¹H NMR (400 MHz, MeOD-d₄) δ 7.85-7.78 (m, 2H), 7.69 (d, J=8.2 Hz, 1H), 7.44-7.36 (m, 2H), 7.36-7.31 (m, 1H), 7.22-7.02 (m, 6H), 5.54 (dd, J=1.5, 8.3 Hz, 1H), 4.11-4.01 (m, 2H), 3.70 (dd, J=8.4, 11.3 Hz, 1H), 3.53 (dd, J=1.6, 11.3 Hz, 1H); ¹³C NMR (100 MHz, MeOD-d₄) δ 174.23, 158.34, 138.93, 135.26, 134.80, 134.07, 133.60, 133.28, 131.11 (broad), 130.53 (broad), 129.64, 129.28 (2C, broad), 128.75, 128.22, 128.06, 126.97, 126.58 (2C, splitted), 125.35, 124.09, 119.16, 68.47, 34.04, 32.36.

Compounds 49-51 were synthesized according to 34 and 35.

### 8. Tetrazole Synthesis:

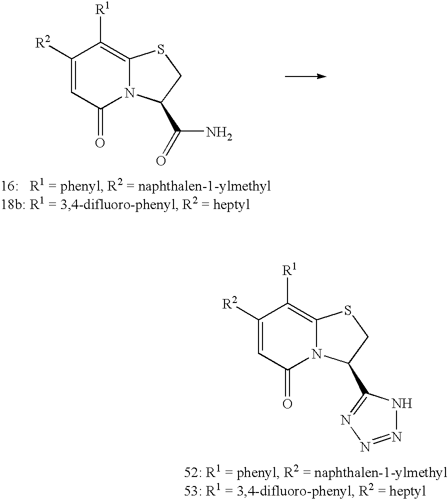

### General Method for Tetrazol Synthesis:

### (3R)-7-Naphthalen-2-ylmethyl-8-phenyl-3-(1H-tetrazol-5-yl)-2,3-dihydro-thiazolo[3,2-a]pyridin-5-one (52)

Prepared as described for 53; Starting from 16 (100 mg, 0.24 mmol) gave 52 as a pale yellow foam (19 mg, 18%) after purification by silica gel chromatography. (MeOH:CH₂Cl₂, 1:4→MeOH). [α]D-9 (c 0.3, DMSO); IR v/cm-1 3062, 2933, 1634, 1562, 1482; ¹H NMR (400 MHz, MeOD-d₄) δ 7.82 (m, 1H), 7.74 (d, J=8.2 Hz, 1H), 7.68 (m, 1H), 7.56-7.32 (m, 8H), 7.28 (d, J=7.0 Hz, 1H), 6.50 (d, J=7.6 Hz, 1H), 5.72 (s, 1H), 4.11-3.91 (m, 3H), 3.34 (m, 1H); ¹³C NMR (100 MHz, MeOD-d₄) δ 163.30, 161.01, 156.59, 150.18, 137.96, 135.40, 135.38, 133.11, 131.78, 130.97, 130.06, 129.95, 129.69, 129.46, 129.00, 128.66, 127.21, 126.71, 126.52, 124.96, 118.84, 115.07, 60.70, 37.71, 36.05; HRMS (FAB+) calcd for [M+Na]⁺ C₂₂H₁₉N₅NaOS 424.1208, obsd 424.1211.

### (3R)-8-(3,4-Difluoro-phenyl)-7-heptyl-3-(1H-tetrazol-5-yl)-2,3-dihydro-thiazolo[3,2-a]pyridin-5-one (53)

18b (50 mg, 0.12 mmol) was dissolved in MeCN (2 ml) and NaN₃ (24 mg, 0.37 mmol) was added. Then SiCl₄ (14 μl, 0.12 mmol) was added dropwise at room temperature. The reaction mixture was heated to reflux overnight. If starting material remained, additional NaN₃ and SiCl₄ were added under maintained refluxing. The reaction mixture was allowed to reach room temperature and was then diluted with EtOAc and washed with aq. Na₂CO₃ and H₂O. The combined aqueous layers were reextracted with EtOAc. The combined organic layers were dried, filtrated and concentrated. Purification by silica gel chromatography (EtOAc:MeOH, 8:2) gave 53 (19 mg, 36%). ¹H NMR (400 MHz, MeOD-d₄) δ 7.47-7.14 (m, 3H), 6.58 (d, J=8.0 Hz, 1H), 6.19 (s, 1H), 3.99 (m, 1H), 3.35-3.32 (m, 1H), 2.33 (t, J=7.4 Hz, 2H), 1.42-1.24 (m, 10H), 0.86 (t, J=7.2 Hz, 3H); ¹³C NMR (100 MHz, MeOD-d₄) δ 163.46, 161.11, 157.94, 152.93, 150.48, 135.46, 128.57, 120.75, 118.82, 118.64, 116.78, 114.31, 60.78, 36.21, 34.16, 32.64, 30.27, 30.18, 29.78, 23.60, 14.31.

### 9. Derivatives of Amine:

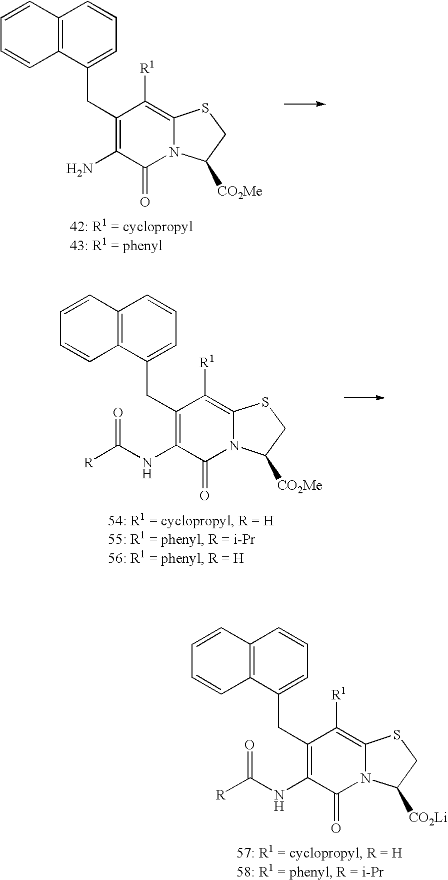

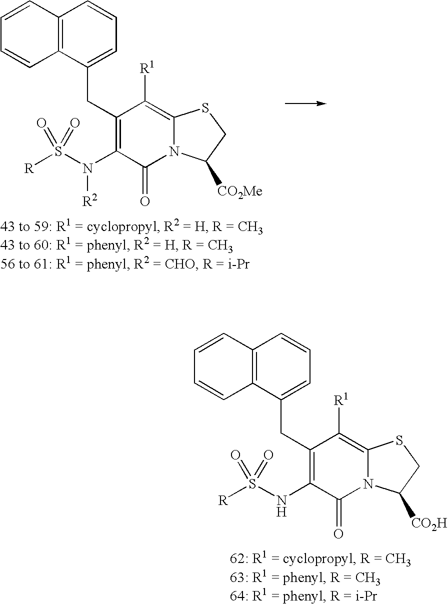

### (3R)-8-Cyclopropyl-6-formylamino-7-naphthalen-1-ylmethyl-5-oxo-2,3-dihydro-5H-thiazolo[3,2-a]pyridine-3-carboxylic acid methyl ester (54)

42 (250 mg, 0.62 mmol) was dissolved in 1.0 ml pyridine:CH₂Cl₂ (1:1). Then formyl acetic anhydride solution (0.4 ml, 5.0 mmol) was added (prepared as described in general experimentals). The solution was stirred at rt overnight and then 10% citric acid (aq.). was added and the aqueous layer was extracted with CH₂Cl₂. The combined organic layers were dried over Na₂SO₄ (s), filtrated, and concentrated. Purification with silica gel chromatography (heptane:EtOAc:MeOH, 50:50:1→5:20:1) gave 54 (248 mg, 93%). [α]_{D}-200 (c 0.5 in CHCl₃); IR v/cm⁻¹ 2360, 2329, 1743, 1674, 1633, 1581, 1501, 1435, 1398, 1342, 1248, 1215, 1170, 1025, 960, 792, 755, 665; As a mixture of rotamers ˜7:5 ¹H NMR (400 MHz, CDCl₃) δ 8.46 (d, J=11.3 Hz, 0.4H), 8.17-8.06 (m, 1.5H), 7.92-7.83 (m, 1H), 7.79-7.68 (m, 1H), 7.64-7.48 (m, 2H), 7.35-7.28 (m, 1H), 7.10 (s, 0.5H), 6.88 (d, J=7.0 Hz, 0.6H), 6.82 (d, J=6.8 Hz, 0.4H), 6.77 (d, J=11.2 Hz, 0.3H), 5.75-5.69 (m, 0.4H), 5.67-5.61 (m, 0.6H), 4.71-4.52 (m, 2H), 3.86 (s, 1.2H), 3.81 (s, 1.8H), 3.77-3.65 (m, 1H), 3.60-3.50 (m, 1H), 1.49-1.40 (m, 0.4H), 1.40-1.31 (m, 0.6H), 0.76-0.64 (m, 2H), 0.64-0.51 (m, 2H); ¹³C NMR (100 MHz, CDCl₃) δ 168.32 (maj), 168.21 (min), 164.35 (min), 160.22 (maj), 158.08, 151.14 (maj), 146.15 (min/maj), 145.99 (maj/min), 144.71 (min), 134.05 (maj), 133.87 (min), 133.67 (maj), 132.38 (min), 131.79 (maj), 131.69 (min), 128.94 (min), 128.74 (maj), 127.79 (min), 126.99 (maj), 126.57 (min), 126.20, 125.79 (maj), 125.45, 123.96 (maj), 123.27, 122.85 (min), 122.37 (min), 120.97 (maj), 114.17 (maj), 113.76 (min), 63.45 (splitted), 53.44 (min), 53.36 (maj), 32.24 (maj), 31.75 (min), 31.66 (maj), 31.60 (min), 11.83 (maj), 11.64 (min), 7.60 (maj), 7.46, 7.06 (min); HRMS (FAB+) calcd for [M+H]⁺ C₂₄H₂₃N₂O₄S 435.1379, obsd 435.1386.

### (3R)-6-Isobutyrylamino-7-naphthalen-1-ylmethyl-5-oxo-8-phenyl-2,3-dihydro-5H-thiazolo[3,2-a]pyridine-3-carboxylic acid methyl ester (55)

Compound 43 (50 mg, 0.11 mmol) was dissolved in 0.2 ml pyridine. Then isobutyryl chloride (13 μl, 0.12 mmol) was added and the solution was stirred at rt for 5 h before heating it to 45° C. overnight. Additional isobutyryl chloride (2 μl) was added together with 0.5 ml CH₂Cl₂ and stirred at rt was continued for 6 h. CH₂Cl₂ was added and the organic layer was washed with 10% citric acid (aq). The combined aqueous layers were reextracted with CH₂Cl₂. The combined organic layers were dried over Na₂SO₄ (s), filtered and concentrated. Purification with silica gel chromatography (heptane:EtOAc:MeOH, 20:40:110:30:1) gave 55 (51 mg, 81%). [α]_{D}-140 (c 0.5, CHCl₃); IR v/cm⁻¹ 2969, 2933, 1746, 1679, 1633, 1585, 1488, 1442, 1398, 1366, 1211, 1156, 746, 705; ¹H NMR (400 MHz, CDCl₃) δ 7.80-7.70 (m, 2H), 7.64 (d, J=8.1 Hz, 1H), 7.44-7.34 (m, 2H), 7.29-7.16 (m, 4H), 7.16-7.08 (m, 1H), 7.01 (d, J=7.1 Hz, 1H), 6.92 (d, J=7.0 Hz, 1H), 6.87 (s, 1H), 5.69 (dd, J=2.1, 8.4 Hz, 1H), 4.32-4.09 (m, 2H), 3.86 (s, 3H), 3.67 (dd, J=8.7, 11.7 Hz, 1H), 3.47 (dd, J=2.3, 11.8 Hz, 1H), 2.36-2.24 (m, 1H), 0.96 (d, J=7.0 Hz, 3H), 0.91 (d, J=7.0 Hz, 3H); ¹³C NMR (100 MHz, CDCl₃) δ 176.11, 168.28, 158.52, 148.19, 144.07, 136.34, 134.38, 133.46, 131.59, 129.76, 129.56, 128.62 (2C, splitted), 128.44, 128.15, 126.72, 125.76, 125.44, 125.24, 124.84, 123.31, 122.30, 116.84, 64.11, 53.38, 35.64, 33.10, 31.68, 19.22 (2C); HRMS (FAB+) calcd for [M+H]⁺ C₃₀H₂₉N₂O₄S 513.1848, obsd 513.1857.

### (3R)-6-Formylamino-7-naphthalen-1-ylmethyl-5-oxo-8-phenyl-2,3-dihydro-5H-thiazolo[3,2-a]pyridine-3-carboxylic acid methyl ester (56)

By following the procedure described for the preparation of 54 from 42, 43 (250 mg, 0.56 mmol) in 1.0 ml pyridine:CH₂Cl₂ (1:1), and formyl acetic anhydride solution (0.35 ml, 4.4 mmol) gave 56 (243 mg, 88%) after purification with silica gel chromatography (heptane:EtOAc:MeOH, 20:40:1→5:20:1). [α]_{D}-158 (c 0.5 in CHCl₃); IR v/cm⁻¹ 1745, 1681, 1633, 1584, 1486, 1433, 1216, 1155, 770, 696; As mixture of rotamers ˜7:4 ¹H NMR (400 MHz, CDCl₃) δ 8.54 (d, J=11.4 Hz, 0.3H), 8.1 (s, 0.6H), 7.86-7.60 (m, 3H), 7.59-7.31 (m, 3H), 7.31-6.91 (m, 7H), 5.77 (d, J=8.2 Hz, 0.4H), 5.65 (dd, J=8.5, 2.0 Hz, 0.6H), 4.28-4.08 (m, 2H), 3.86 (s, 1H), 3.76 (s, 2H), 3.76-3.57 (m, 1H), 3.54-3.39 (m, 1H); ¹³C NMR (100 MHz, CDCl₃) δ 168.04, 164.41 (min), 160.25 (maj), 158.09, 148.84 (maj), 145.27 (maj), 144.17 (min), 143.67 (min), 135.92 (maj), 135.59 (min), 133.80 (maj), 133.57 (min), 133.35 (maj), 132.29 (min), 131.41 (maj), 131.26 (min), 129.64, 129.33 (maj), 128.98 (min), 128.74-128.28 (m, 3C+1 min C), 128.12 (maj), 127.65 (min), 126.76 (maj), 126.21 (min), 125.88 (min), 125.78 (maj), 125.43 (maj), 125.23, 124.72 (maj), 123.85 (min), 123.09 (maj), 122.69 (min), 122.18 (min), 120.91 (maj), 116.58 (maj), 116.36 (min), 64.27 (min), 64.12 (maj), 53.38 (min), 53.25 (maj), 32.91 (maj), 32.33 (min), 31.53; HRMS (FAB+) calcd for [M+H]⁺ C₂₇H₂₃N₂O₄S 471.1379, obsd 471.1382.

### (3R)-8-Cyclopropyl-6-formylamino-7-naphthalen-1-ylmethyl-5-oxo-2,3-dihydro-5H-thiazolo[3,2-a]pyridine-3-lithium carboxylate (57)

[α]_{D}-138 (c 0.3, DMSO); IR v/cm⁻¹ 1610, 1562, 1501, 1472, 1395, 1378, 1273, 1250, 775, 661; As a mixture of rotamers ˜7:3 ¹H NMR (400 MHz, MeOD-d₄) δ 8.22-8.14 (m, 1H), 8.07 (s, 0.3H), 8.02 (s, 0.6H), 7.90-7.84 (m, 1H), 7.75-7.68 (m, 1H), 7.61-7.47 (m, 2H), 7.36-7.28 (m, 1H), 7.02 (dd, J=0.7, 7.1 Hz, 0.7H), 7.00-6.96 (m, 0.3H), 5.53-5.48 (m, 1H), 4.72-4.52 (m, 2H), 3.82-3.73 (m, 1H), 3.66-3.59 (m, 1H), 1.43-1.27 (m, 1H), 0.66-0.47 (m, 4H); ¹³C NMR (100 MHz, MeOD-d₄) δ 173.93 (maj), 173.88 (min), 168.19 (min), 163.38 (maj), 160.98 (min), 160.05 (maj), 153.24 (maj), 151.38 (min), 150.55 (maj), 150.16 (min), 135.27, 135.21 (maj), 134.97 (min), 133.29 (splitted), 129.83 (min), 129.79 (maj), 128.17 (min), 127.86 (maj), 127.33 (min), 127.18 (maj), 126.89 (min), 126.71 (maj), 126.64 (maj), 126.55 (min), 125.60 (maj), 125.27 (min), 124.12, 122.49 (min), 121.68 (maj), 115.14 (min), 115.08 (maj), 68.07 (min), 67.96 (maj), 34.03, 32.65 (maj), 32.36 (min), 12.70 (maj), 12.63 (min), 8.11 (broad), 7.75 (maj), 7.63 (min).

### (3R)-6-Isobutyrylamino-7-naphthalen-1-ylmethyl-5-oxo-8-phenyl-2,3-dihydro-5H-thiazolo[3,2-a]pyridine-3-lithium carboxylate (58)

[α]_{D}-132 (c 0.4, DMSO); IR v/cm⁻¹ 1617, 1568, 1492, 1440, 1393, 1291, 770; ¹H NMR (400 MHz, MeOD-d₄) δ 7.80-7.75 (m, 1H), 7.75-7.69 (m, 1H), 7.65 (d, J=8.2 Hz, 1H), 7.42-7.33 (m, 2H), 7.33-7.28 (m, 1H), 7.18-7.07 (m, 6H), 5.55 (dd, J=1.6, 8.7 Hz, 1H), 4.15-4.04 (m, 2H), 3.77 (dd, J=8.7, 11.4 Hz, 1H), 3.56 (dd, J=1.6, 11.4 Hz, 1H), 2.42-2.31 (m, 1H), 0.92 (d, J=6.9 Hz, 3H), 0.74 (d, J=6.9 Hz, 3H); ¹³C NMR (100 MHz, MeOD-d₄) δ 179.65, 173.90, 160.69, 150.82, 149.10, 138.14, 135.18, 134.96, 132.92, 131.31, 130.83, 129.57, 129.48 (2C), 129.08, 127.70, 126.88, 126.56, 126.45, 126.25, 123.86, 122.88, 117.81, 68.71, 36.05, 34.11, 33.07, 19.80, 19.37.

### (3R)-8-Cyclopropyl-6-methanesulfonylamino-7-naphthalen-1-ylmethyl-5-oxo-2,3-dihydro-5H-thiazolo[3,2-a]pyridine-3-carboxylic acid methyl ester (59)

42 (60 mg, 0.15 mmol) was dissolved in 0.2 ml pyridine and methane sulfonyl chloride (23 μl, 0.30 mmol) was added. After 5 h CH₂Cl₂ was added and the organic layer was washed with 10% citric acid (aq.). The combined aqueous layers were reextracted with CH₂Cl₂. The combined organic layers were dried over Na₂SO₄ (s), filtrated and concentrated. Purification with silica gel chromatography (heptane:EtOAc:MeOH, 50:50:1→20:40:1) gave 59 (63 mg, 88%). [α]_{D}-160 (c 0.5, CHCl₃); IR v/cm⁻¹ 1745, 1631, 1581, 1498, 1434, 1392, 1317, 1243, 1218, 1149, 973, 779; ¹H NMR (400 MHz, CDCl₃) δ 8.20 (d, J=8.9 Hz, 1H), 7.85 (d, J=8.1 Hz, 1H), 7.70 (d, J=8.2 Hz, 1H), 7.60-7.47 (m, 2H), 7.32-7.26 (m, 1H), 6.83 (d, J=7.1 Hz, 1H), 6.47 (s, 1H), 5.70 (dd, J=1.5, 8.5 Hz, 1H), 5.00-4.76 (m, 2H), 3.82 (s, 3H), 3.70 (dd, J=8.9, 12.1 Hz, 1H), 3.53 (dd, J=1.6, 12.0 Hz, 1H), 3.03 (s, 3H), 1.22-1.13 (m, 1H), 0.71-0.52 (m, 4H); ¹³C NMR (100 MHz, CDCl₃) δ 167.99, 159.09, 155.40, 147.29, 134.21, 133.68, 131.85, 128.64, 126.93, 126.16, 125.74, 125.24, 124.30, 123.49, 121.43, 114.73, 63.46, 53.41, 41.08, 31.69, 31.54, 12.03, 7.80, 7.39; HRMS (FAB+) calcd for [M+H]⁺ C₂₄H₂₅N₂O₅S₂ 485.1205, obsd 485.1209.

### (3R)-6-Methanesulfonylamino-7-naphthalen-1-ylmethyl-5-oxo-8-phenyl-2,3-dihydro-5H-thiazolo[3,2-a]pyridine-3-carboxylic acid methyl ester (60)

By following the procedure described for the preparation of 59 from 42, 43 (60 mg, 0.14 mmol) in pyridine (0.2 ml), and methane sulfonyl chloride (21 μl, 0.27 mmol) gave 60 (58 mg, 84%) after purification with silica gel chromatography (heptane:EtOAc, 1:3). [α]_{D}-125 (c 0.5, CHCl₃); IR v/cm⁻¹ 1751, 1631, 1577, 1494, 1442, 1390, 1319, 1216, 1145, 975, 777, 700; ¹H NMR (400 MHz, CDCl₃) δ 7.78-7.71 (m, 2H), 7.63 (d, J=8.1 Hz, 1H), 7.43-7.32 (m, 2H), 7.23-7.17 (m, 1H), 7.16-7.09 (m, 2H), 7.08-6.98 (m, 2H), 6.87 (d, J=7.9 Hz, 1H), 6.79 (d, J=7.0 Hz, 1H), 6.55 (s, 1H), 5.74 (dd, J=8.7, 2.2 Hz, 1H), 4.65-4.33 (m, 2H), 3.83 (s, 3H), 3.67 (dd, J=8.7, 11.8 Hz, 1H), 3.45 (dd, J=2.1, 11.8 Hz, 1H), 3.11 (s, 3H); ¹³C NMR (100 MHz, CDCl₃) δ 167.88, 159.20, 153.03, 146.74, 135.79, 133.97, 133.48, 131.60, 129.77, 129.31, 128.54, 128.47, 128.35, 128.23, 126.90, 125.86, 125.47, 125.44, 125.06, 123.41, 121.27, 117.10, 64.31, 53.48, 41.31, 32.60, 31.57; HRMS (FAB+) calcd for [M+H]⁺ C₂₇H₂₅N₂O₅S₂ 521.1205, obsd 521.1212.

### (3R)-6-[Formyl-(propane-2-sulfonyl)-amino]-7-naphthalen-1-ylmethyl-5-oxo-8-phenyl-2,3-dihydro-5H-thiazolo[3,2-a]pyridine-3-carboxylic acid methyl ester (61)

56 (60 mg, 0.13 mmol) was dissolved in 1.5 ml THF and the solution was cooled to −42° C. Then t-BuOK (0.95 M in THF, 135 μl, 0.13 mmol) was added dropwise. After stirring at −42° C. for 45 min., isopropyl sulfonyl chloride (43 μl, 0.38 mmol) was added. After 1.5 h the temperature was allowed to reach rt and the reaction was stirred overnight. Saturated NaHCO₃ (aq.) and brine was added and the product was extracted with CH₂Cl₂. The combined organic layers were dried over Na₂SO₄ (s), filtrated, and concentrated. Purification with silica gel chromatography (heptane:EtOAc:MeOH, 130:70:1→20:40:1) gave 61 (30 mg, 41%): [α]_{D} 0 (c 0.5, CHCl₃); IR v/cm⁻¹ 2923, 1750, 1710, 1644, 1582, 1488, 1441, 1398, 1346, 1261, 1214, 1145, 1114, 1016, 941, 793, 748, 695; As a mixture of rotamers ˜3:2 ¹H NMR (400 MHz, CDCl₃) δ 8.75 (s, 0.3H), 8.70 (s, 0.6H), 7.74-7.57 (m, 3H), 7.39-7.08 (m, 6H), 7.02-6.92 (m, 1H), 6.80-6.74 (m, 1H), 6.62-6.53 (m, 1H), 5.76-5.70 (m, 1H), 4.58-4.06 (m, 2H), 3.89 (s, 1.3H), 3.84 (s, 1.8H), 3.78-3.67 (m, 1.5H), 3.62-3.53 (m, 0.6H), 3.54-3.46 (m, 1.1H), 1.60-1.47 (m, 6H); ¹³C NMR (100 MHz, CDCl₃) δ 167.75, 161.62 (maj), 161.14 (min), 158.03 (min), 157.67 (min), 157.52 (maj), 157.44 (maj), 150.05 (maj), 149.81 (min), 135.38 (min), 135.31 (maj), 133.34 (maj), 133.26 (maj), 133.19 (2 min C), 131.41 (maj), 131.34 (min), 129.92 (maj), 129.86 (min), 129.42 (maj), 129.16 (min), 128.48 (maj), 128.42 (min), 128.28 (maj), 128.25 (min), 128.17 (maj), 128.12 (splitted), 127.99 (min), 126.93 (maj), 126.88 (min), 126.72, 125.68 (maj), 125.54 (min), 125.26, 125.19 (min), 125.10 (maj), 123.05 (maj), 123.00 (min), 118.88 (min), 118.60 (maj), 116.85 (maj), 116.69 (min), 64.42 (maj), 64.22 (min), 56.76 (min), 56.46 (maj), 53.63 (min), 53.38 (maj), 32.50 (maj), 32.44 (min), 31.75 (min), 31.66 (maj), 16.87 (maj), 16.76 (min), 15.82 (maj), 15.73 (min); HRMS (FAB+) calcd for [M+H]⁺ C₃₀H₂₉N₂O₆S₂ 577.1467, obsd 577.1475.

### (3R)-8-Cyclopropyl-6-methanesulfonylamino-7-naphthalen-1-ylmethyl-5-oxo-2,3-dihydro-5H-thiazolo[3,2-a]pyridine-3-carboxylic acid (62)

Hydrolysis of 59 required additional 0.5 eq. LiOH and the residue was therefore treated with Amberlite® IR120+ ion-exchange resin to yield 62. [α]_{D}-144 (c 0.5, DMSO); IR v/cm⁻¹ 1729, 1625, 1498, 1392, 1313, 1244, 1144, 970, 777; ¹H NMR (400 MHz, MeOD-d₄) δ 8.21 (d, J=8.4 Hz, 1H), 7.85 (d, J=8.1 Hz, 1H), 7.70 (d, J=8.2 Hz, 1H), 7.58-7.52 (m, 1H), 7.51-7.45 (m, 1H), 7.32-7.26 (m, 1H), 6.89 (d, J=7.1 Hz, 1H), 5.70 (dd, J=1.4, 8.8 Hz, 1H), 4.94-4.77 (m, 2H), 3.85 (dd, J=8.9, 12.0 Hz, 1H), 3.60 (dd, J=1.5, 12.0 Hz, 1H), 3.02 (s, 3H), 1.17-1.08 (m, 1H), 0.64-0.48 (m, 4H); ¹³C NMR (100 MHz, MeOD-d₄) δ 170.86, 160.95, 157.46, 150.11, 135.77, 135.23, 133.31, 129.74, 127.85, 127.16, 126.73, 126.41, 125.74, 124.40, 122.54, 115.62, 65.23, 41.95, 32.73, 32.56, 13.01, 8.57, 8.14.

### (3R)-6-Methanesulfonylamino-7-naphthalen-1-ylmethyl-5-oxo-8-phenyl-2,3-dihydro-5H-thiazolo[3,2-a]pyridine-3-carboxylic acid (63)

Hydrolysis of 60 required additional 0.5 eq. LiOH and the residue was therefore treated with Amberlite® IR120+ ion-exchange resin to yield 63. [α]_{D}-106 (c 0.5, DMSO); IR v/cm⁻¹ 1749, 1624, 1497, 1385, 1311, 1161, 1112, 775; ¹H NMR (400 MHz, MeOD-d₄) δ 7.77-7.73 (m, 1H), 7.69 (d, J=8.3 Hz, 1H), 7.62 (d, J=8.1 Hz, 1H), 7.40-7.30 (m, 2H), 7.25-7.19 (m, 1H), 7.11-7.05 (m, 2H), 7.04-6.97 (m, 1H), 6.96-6.90 (m, 1H), 6.87-6.81 (m, 2H), 5.76 (dd, J=1.7, 8.9 Hz, 1H), 4.55-4.33 (m, 2H), 3.90-3.82 (m, 1H), 3.56 (dd, J=1.7, 11.9 Hz, 1H), 3.09 (s, 3H); ¹³C NMR (100 MHz, MeOD-d₄) δ 170.82, 161.11, 155.17, 149.52, 137.66, 135.48, 135.00, 133.00, 131.07, 128.80, 129.51, 129.47, 129.43, 129.12, 127.75, 126.83 (2C, broad), 126.42, 126.22, 124.23, 122.40, 118.10, 66.06, 42.03, 33.42, 32.65.

### (3R)-7-Naphthalen-1-ylmethyl-5-oxo-8-phenyl-6-(propane-2-sulfonylamino)-2,3-dihydro-5H-thiazolo[3,2-a]pyridine-3-carboxylic acid (64)

61 (28 mg, 0.049 mmol) was dissolved in 1 ml THF:MeOH (3:7) and 0.1 M LiOH (aq.) (0.975 ml, 0.098 mmol) was added dropwise at 0° C. The solution was allowed to attain rt while stirred overnight and was then concentrated, first from EtOH and then MeOH. This gave the lithium carboxylate of 64 together with the lithium formate from the deprotection. To remove the lithium formate, the residue was dissolved in 2.0 ml MeOH:CH₂Cl₂ (3:1) and swirled with a small spoon of Amberlite® IR120+ ion-exchange resin. The resin was removed by filtration and the filtrate was concentrated to give 64 (26 mg, quant.). [α]_{D} 0 (c 0.5, MeOH); IR v/cm⁻¹ 1736, 1625, 1493, 1256, 1132; ¹H NMR (400 MHz, MeOD-d₄) δ 7.76-7.73 (m, 1H), 7.69 (d, J=8.2 Hz, 1H), 7.62 (d, J=8.2 Hz, 1H), 7.39-7.29 (m, 2H), 7.25-7.19 (m, 1H), 7.09-6.98 (m, 3H), 6.91-6.85 (m, 3H), 5.72 (dd, J=1.7, 8.9 Hz, 1H), 4.51-4.39 (m, 2H), 3.81 (dd, J=8.9, 12.0 Hz, 1H), 3.53 (dd, J=1.7, 12.0 Hz, 1H), 3.49-3.38 (m, 1H), 1.44 (t, J=6.9 Hz, 6H); ¹³C NMR (100 MHz, MeOD-d₄) δ 170.78, 161.33, 155.25, 149.14, 137.72, 135.46, 134.96, 132.98, 131.07, 130.78, 129.44 (3C, splitted), 129.05, 127.74, 126.92, 126.81, 126.40, 126.23, 124.21, 122.81, 118.05, 65.94, 55.99, 33.65, 32.63, 17.16, 17.08; HRMS (FAB+) calcd for [M+H]⁺ C₂₈H₂₇N₂O₅S₂ 535.1361, obsd 535.1360.

### 10. Synthesis of the Acyl Sulfonamide:

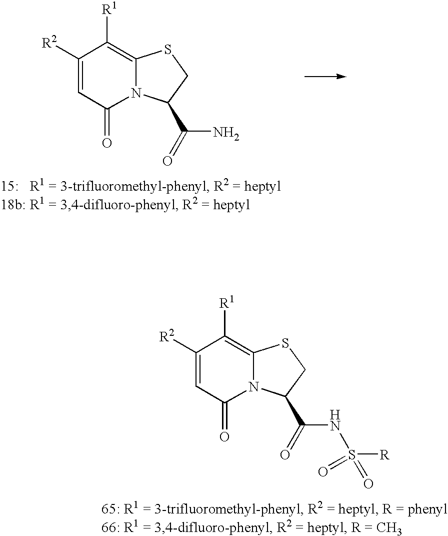

### General Procedure for the Synthesis of the Acyl Sulfonamide Derivatives:

### (3R)—N-[7-Heptyl-5-oxo-8-(3-trifluoromethyl-phenyl)-2,3-dihydro-5H-thiazolo[3,2-a]pyridine-3-carbonyl]-benzenesulfonamide (65)

15 (24 mg, 0.05 mmol) was suspended in CH₂Cl₂ (1 ml) in a microwave vial. To the suspension was added N,N′-carbonyldiimidazole (27 mg, 0.16 mmol) in one portion at room temperature. After 1 hour and 45 minutes, benzene sulfonamide (34 mg, 0.21 mmol) was added in one portion at rt. The vial was capped and heated with microwave irradiation (normal absorption) at 80° C. for 7 hours. The solution was diluted with CH₂Cl₂ and washed with 5% aqueous citric acid. The combined aqueous layers were reextracted with CH₂Cl₂ and the combined organic layers were dried, filtrated and concentrated. Purification by silica gel chromatography (EtOAc:MeOH, 9:1) gave 65 (17.4 mg, 62%). ¹H NMR (400 MHz, CDCl₃) δ 8.11-8.04 (m, 3H), 7.68-7.52 (m, 6H), 5.61 (d, J=7.56 Hz, 1H), 3.87 (d, J=11.34 Hz, 1H), 3.48 (m, 1H), 2.22 (t, J=7.56 Hz, 2H), 1.39-1.14 (m, 10H), 0.84 (t, J=7.09 Hz, 3H).

### (3R)—N-[8-(3,4-Difluoro-phenyl)-7-heptyl-5-oxo-2,3-dihydro-5H-thiazolo[3,2-a]pyridine-3-carbonyl]-methanesulfonamide (66)

18b (75 mg, 0.18 mmol) was suspended in CH₂Cl₂ (1 mL) in a microwave vial. To the suspension was added N,N′-carbonyldiimidazole (90 mg, 0.553 mmol) in one portion at room temperature. After 1 hour and 45 minutes, methane sulfonamide (70 mg, 0.736 mmol) was added in one portion at rt. The vial was capped and heated with microwave irradiation (normal absorption) at 80° C. for 7 hours. The solution was diluted with CH₂Cl₂ and washed with 5% aqueous citric acid. The combined aqueous layers were reextracted with CH₂Cl₂ and the combined organic layers were dried, filtrated and concentrated. Purification by silica gel chromatography (EtOAc→EtOAc:MeOH, 9:1) gave 66 (58 mg, 65%). ¹H NMR (400 MHz, MeOD-d4) δ 7.41-7.34 (m, 1H), 7.28-7.24 (m, 1H), 7.12-7.13 (m, 1H), 6.21 (s, 1H), 5.54 (m, 1H), 4.86 (bs, 1H), 3.87-3.81 (m, 1H), 3.54-3.51 (m, 1H), 3.33 (s, 3H), 2.32 (t, J=6.79 Hz, 2H), 1.40-1.36 (m, 2H), 1.25-1.17 (m, 8H), 0.86 (t, J=7.64 Hz, 3H); ¹³C NMR (100 MHz, MeOD-d4) δ 168.71, 163.47, 158.81, 152.81, 150.72, 150.33, 134.84, 128.40, 120.55, 119.00, 116.39, 113.94, 66.44, 41.39, 34.14, 32.65, 31.97, 30.23, 30.09, 29.76, 23.61, 14.34.

### 11. Desulfurization:

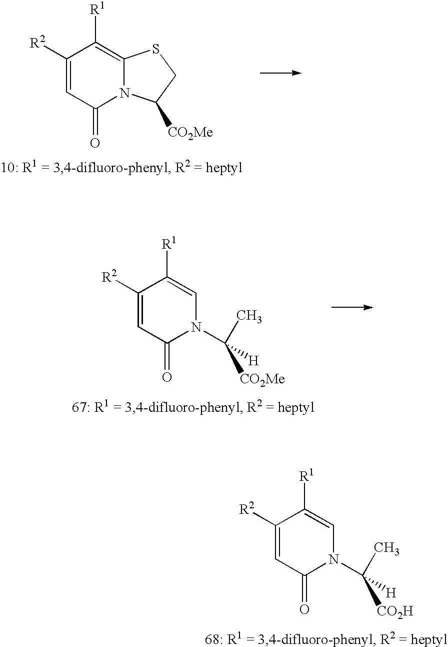

### General Procedure for Desulfurization:

### (2S)-2-[5-(3,4-Difluoro-phenyl)-4-heptyl-2-oxo-2H-pyridin-1-yl]-propionic acid methyl ester (67)

10 (31 mg, 0.07 mmol) was dissolved in MeOH (10 mL). Raney® 2800 nickel (water slurry, 300 mg) was added at room temperature and the reaction mixture was heated to reflux. After 2+2 hours the reaction was fed with two additional portions of Raney® 2800 nickel (water slurry, 300+300 mg). Addition was carried out under continuous refluxing. The reaction was monitored by LC-MS to avoid reduction of the 2-pyridone ring. After a total of 7 hours, the reaction mixture was allowed to reach room temperature and filtrated through a pad of Celite. The solid phase was washed with CH₂Cl₂:MeOH (4:1). The filtrate was concentrated and purification by silica gel chromatography (heptane:EtOAc, 2:3) gave 67 (27 mg, 95%).

### (2S)-2-[5-(3,4-Difluoro-phenyl)-4-heptyl-2-oxo-2H-pyridin-1-yl]-propionic acid (68)

1.5 ml MeOH was added to 67 (18 mg, 0.04 mmol) and allowed to stir for five minutes. Then 0.1 M LiOH (aq.) (0.4 ml) was added and the solution was stirred over-night. Then concentrated and co-concentrated three times with ethanol. The mixture was dissolved in 1.5 ml MeOH then added Amberlite® IR-120+(500 mg) and stirred well for 10 min. The solid phase was washed with MeOH. The filtrate was concentrated from CH₂Cl₂ gave 68 as a solid (17.2 mg, quant.) ¹H NMR (400 MHz, MeOD-d4) δ 7.48 (s, 1H), 7.36-7.26 (m, 2H), 7.15-7.12 (m, 1H), 6.47 (s, 1H), 5.33 (dd, J=7.39, 14.48 Hz, 1H), 2.47 (t, J=7.78 Hz, 2H), 1.66 (d, J=7.32 Hz, 3H), 1.48-1.16 (m, 10H), 0.86 (t, J=7.18 Hz, 3H); ¹³C NMR (100 MHz, MeOD-d₄) δ 173.99, 163.86, 156.80, 152.57, 150.07, 137.00, 135.22, 127.76, 122.80, 120.06, 118.61, 118.34, 57.12, 33.89, 32.65, 30.10, 30.07, 29.78, 23.58, 16.70, 14.30.

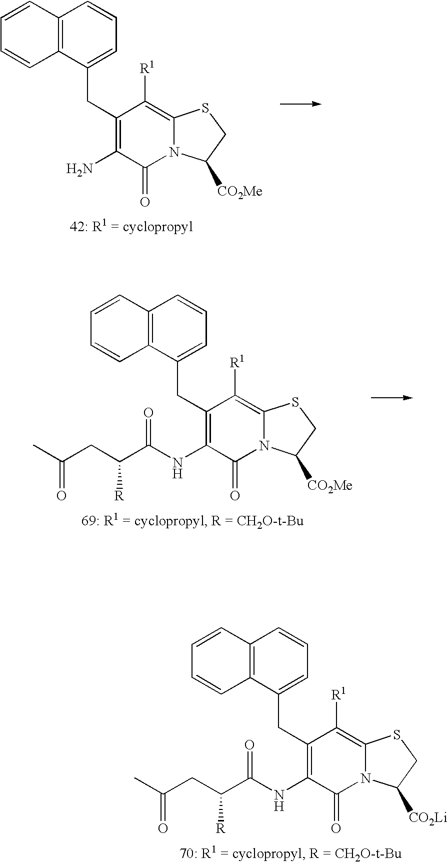

### (3R)-6-(2-Acetylamino-3-tert-butoxy-propionylamino)-8-cyclopropyl-7-naphthalen-1-ylmethyl-5-oxo-2,3-dihydro-5H-thiazolo[3,2-a]pyridine-3-carboxylic acid methyl ester (69)

42 (60 mg, 0.15 mmol), HOAt (24 mg, 0.18 mmol), Ac-Ser(t-Bu)—OH (39 mg, 0.19 mmol), and DIC (28 μl, 0.19 mmol) in 0.8 ml CH₂Cl₂:THF (1:1) (24 h) gave 69 (74 mg, 85%) after purification with silica gel chromatography (heptane:EtOAc:MeOH, 5:20:1→5:40:4). [α]_{D}-184 (c 0.5, CHCl₃); IR v/cm⁻¹ 1747, 1639, 1587, 1496, 1363, 1207, 1081, 1022, 792, 773; As a mixture of rotamers ˜5:4 ¹H NMR (400 MHz, CDCl₃) δ 8.09 (d, J=8.2 Hz, 1H), 8.06 (s, 0.5H), 7.98 (s, 0.4H), 7.84 (d, J=8.1 Hz, 1H), 7.69 (d, J=8.2 Hz, 1H), 7.58-7.46 (m, 2H), 7.33-7.26 (m, 1H), 6.89 (d, J=7.0 Hz, 1H), 6.43 (d, J=6.1 Hz, 0.4H), 6.35 (d, J=6.3 Hz, 0.5H), 5.67 (dd, J=2.2, 8.6 Hz, 1H), 4.62-4.49 (m, 2H), 4.43-4.32 (m, 1H), 3.84-3.80 (m, 3H), 3.73-3.61 (m, 2H), 3.51 (dd, J=2.2, 11.9 Hz, 1H), 3.22 (t, J=8.4 Hz, 0.6H), 3.05 (t, J=8.5 Hz, 0.5H), 1.86 (s, 1.4H), 1.84 (s, 1.5H), 1.35-1.25 (m, 1H), 0.88 (s, 5H), 0.82 (s, 4H), 0.69-0.60 (m, 2H), 0.60-0.51 (m, 2H); ¹³C NMR (100 MHz, CDCl₃) δ 169.97 (splitted), 169.89, 168.39, 168.34, 157.84, 157.79, 150.86, 145.97, 145.78, 133.98, 133.78, 133.69, 131.77, 131.73, 128.73, 126.95, 126.93, 126.06, 125.68, 125.49, 125.46, 123.93, 11.89, 123.11, 122.21, 122.18, 113.60, 113.54, 74.14, 74.10, 63.31, 63.28, 60.98, 60.95, 53.35, 53.27, 53.23, 53.17, 31.92, 31.86, 31.64, 31.58, 27.05-26.85 (3C), 22.95, 22.93, 11.73, 11.68, 7.49-7.24 (m, 2C), 7.34, 7.30; HRMS (FAB+) calcd for [M+Na]⁺ C₃₂H₃₇N₃NaO₆S 614.2301, obsd 614.2310.

### (3R)-6-(2-Acetylamino-3-hydroxy-propionylamino)-8-cyclopropyl-7-naphthalen-1-ylmethyl-5-oxo-2,3-dihydro-5H-thiazolo[3,2-a]pyridine-3-lithium carboxylate (70)

i) 69 (42 mg, 0.071 mmol) was dissolved in 2 ml CH₂Cl₂:TFA (9:1) and stirred at rt overnight. The solution was then concentrated, first from CH₂Cl₂:TFA 9:1, then from THF:MeOH:H₂O and finally from toluene. Purification with silica gel chromatography (heptane:EtOAc:MeOH, 5:40:4→5:60:8) gave the t-butyl deprotected methyl ester (25 mg, 66%). [α]_{D}-155 (c 0.5 in CHCl₃); IR v/cm⁻¹ 2960, 1748, 1629, 1579, 1500, 1436, 1344, 1258, 1200, 1175, 1126, 1064, 1018, 965, 793, 747; As a mixture of rotamers ˜5:4 ¹H NMR (400 MHz, CDCl₃) δ 8.11 (dd, J=8.4, 2.7 Hz, 1H), 8.04-7.94 (m, 1H), 7.85 (dd, J=8.0 Hz, 1H), 7.69 (dd, J=8.1 Hz, 1H), 7.61-7.48 (m, 2H), 7.32-7.25 (m, 1H), 7.05-6.97 (m, 0.5H), 6.90-6.77 (m, 1.5H), 5.74-5.66 (m, 1H), 4.62-4.51 (m, 2H), 4.40-4.26 (m, 1H), 3.85-3.40 (m, 8H), 1.58 (s, 1.7H), 1.52 (s, 1.3H), 1.40-1.30 (m, 1H), 0.71-0.61 (m, 2H), 0.59-0.49 (m, 2H); ¹³C NMR (100 MHz, CDCl₃) δ 171.34, 171.21, 170.94, 170.72, 168.44, 168.40, 158.53, 153.10, 147.32, 133.67, 133.47, 133.42, 131.72, 128.77, 128.70, 127.08, 126.30 (splitted), 125.91, 125.86, 125.56 (splitted), 123.94, 123.80, 123.25, 123.14, 122.24, 122.14, 114.69, 114.60, 63.63, 62.65, 62.37, 55.31, 54.87, 53.47, 53.45, 31.60 (2C), 22.32, 22.13, 11.71, 7.37, 7.23, 7.15; HRMS (FAB+) calcd for [M+Na]⁺ C₂₈H₂₉N₃NaO₆S 558.1675, obsd 558.1678.

ii) The purified product from the t-butyl deprotection was then subjected to alkaline ester hydrolysis according to the general procedure to yield 70.

[α]_{D}-190 (c 0.5, MeOH); IR v/cm⁻¹ 1617, 1570, 1503, 1435, 1377, 1269, 1201, 1179, 1137, 1059, 1034, 792; As a mixture of rotamers ˜5:4 ¹H NMR (400 MHz, MeOD-d₄) δ 8.23-8.16 (m, 1H), 7.86 (d, J=8.1 Hz, 1H), 7.71 (d, J=8.2 Hz, 1H), 7.59-7.54 (m, 1H), 7.53-7.47 (m, 1H), 7.34-7.28 (m, 1H), 7.06-7.01 (m, 1H), 5.51-5.46 (m, 1H), 4.65-4.50 (m, 2H), 4.39 (t, J=5.5 Hz, 0.6H), 4.33 (t, J=5.0 Hz, 0.5H), 3.80-3.55 (m, 4H), 1.74 (s, 1.7H), 1.69 (s, 1.3H), 1.32-1.24 (m, 1H), 0.63-0.44 (m, 4H); ¹³C NMR (100 MHz, MeOD-d₄) δ 173.99, 173.39, 173.26, 172.76, 172.70, 160.36, 160.33, 153.87, 153.83, 150.54, 150.40, 135.45 (splitted), 135.22, 133.31, 129.75, 129.73, 127.85 (splitted), 127.82, 127.17, 126.73, 126.70, 126.64, 125.76, 125.62, 124.40, 124.34, 122.93, 122.68, 115.25, 115.19, 68.02, 63.18, 62.96, 57.33, 56.79, 33.96, 32.53, 32.46, 22.28, 22.22, 12.70 (splitted), 8.11, 8.07, 7.80, 7.71.

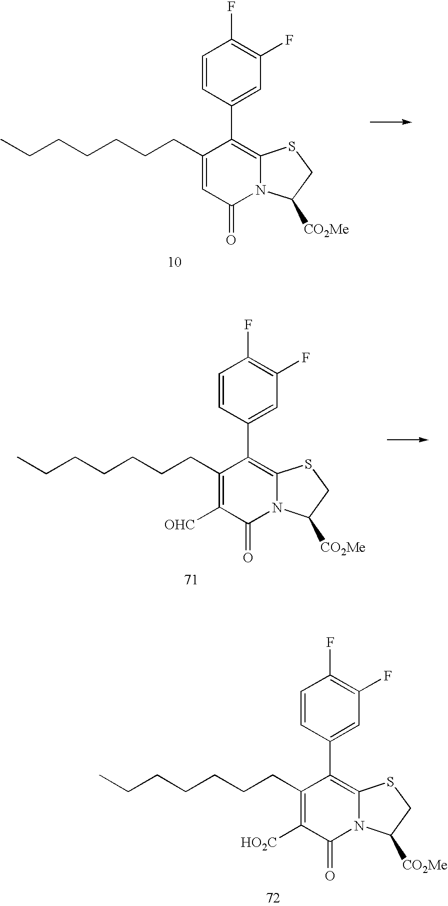

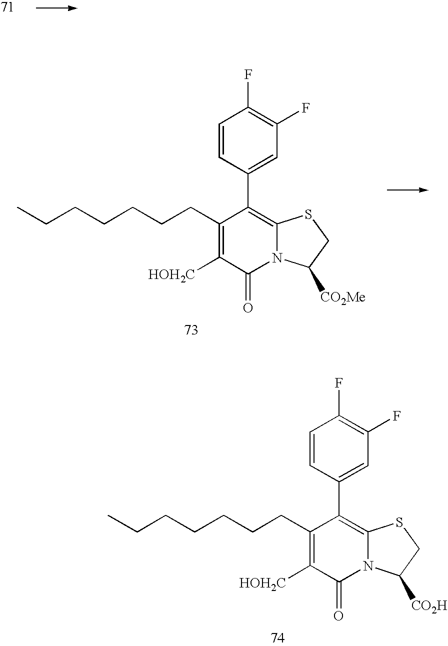

### Aldehyde Synthesis in R³ Position:

### (3R)-8-(3,4-Difluoro-phenyl)-6-formyl-7-heptyl-5-oxo-2,3-dihydro-5H-thiazolo[3,2-a]pyridine-3-carboxylic acid methyl ester (71)

10 (357 mg, 0.85 mmol) was added to a stirred solution of Vilsmeier's salts (Cl⁻ Me₂N⁺═CHCl) (435 mg, 3.4 mmol) in dry CH₂Cl₂ under an inert atmosphere. The solution was heated to reflux (3 h) cooled to room temperature and concentrated. The residue was diluted with CH₂Cl₂ and quenched with salt NaHCO₃ (aq.) extracted with CH₂Cl₂ and concentrated. Purification with silica gel chromatography (heptane: EtOAc, 1:9) gave 71 (302 mg, 79%). ¹³C NMR (100 MHz, CDCl₃) δ 190.92, 167.87. 162.47, 161.65, 155.63, 151.88-151.48 (m, 1C), 149.38-148.98 (m, 1C), 132.28, 127.12-126.66 (m, 1C), 119.90-119.28 (m, 1C), 118.11-117.87 (m, 1C), 117.19, 115.16, 64.01, 53.54, 31.54, 31.44, 30.17, 29.81, 29.72, 28.47, 22.48, 13.94.

### (3R)-8-(3,4-Difluoro-phenyl)-7-heptyl-5-oxo-2,3-dihydro-5H-thiazolo[3,2-a]pyridine-3,6-dicarboxylic acid 3-methyl ester (72)

NaH₂PO₄ (23 mg, 0.17 mmol) was dissolved in H₂O and added drop wise to 71 (38 mg, 0.085 mmol) in DMSO at rt. Then NaClO₂ (31 mg, 0.34 mmol) in water was added drop wise over 30 min. White precipitation, stirred in rt 2 h. Poured into a separatory funnel containing ice-cooled 1 (M) HCl. Water phase extracted with CH₂Cl₂. Combined organic phases concentrated. Dissolved in water:CH₃CN, freeze dried. Dissolved in chloroform and co-concentrated gave 72 (35 mg, 89%). ¹H NMR (400 MHz, CDCl₃) δ 7.627.58 (m, 1H), 7.44-7.42 (m, 1H), 7.34-7.32 (m 1H), 6.10 (d, J=7.2 Hz, 1H), 4.19 (s, 3H), 4.19-4.12 (m, 1H), 3.89-3.86 (m, 1H), 3.24.3.21 (m, 2H), 1.72-1.42 (m, 10H), 1.15 (t, J=7.11 Hz, 3H); ¹³C NMR (100 MHz, CDCl₃) δ 167.39, 165.74, 165.13, 165.11, 164.09, 153.79, 152.02 149.34, 132.42, 126.82, 119.59, 118.32, 110.53, 64.86, 42.77, 32.16, 31.77, 31.61, 30.07, 30.00, 28.61, 22.63, 14.09.

### (3R)-8-(3,4-Difluoro-phenyl)-7-heptyl-6-hydroxymethyl-5-oxo-2,3-dihydro-5H-thiazolo[3,2-a]pyridine-3-carboxylic acid methyl ester (73)

71 (29 mg, 0.06 mmol) was dissolved in dry THF at 0° C. and 2M BH₃.DMS.THF (0.034 mL, 0.06 mmol) was added drop wise. Allowed to stirred at rt 1 h. Quenched with MeOH. Concentrated twice from MeOH. Purification with silica gel chromatography (CH₂Cl₂: MeOH, 9:1) gave 73 (22 mg, 75%).

Compound 74 was synthesized according to 12.

### Bromination:

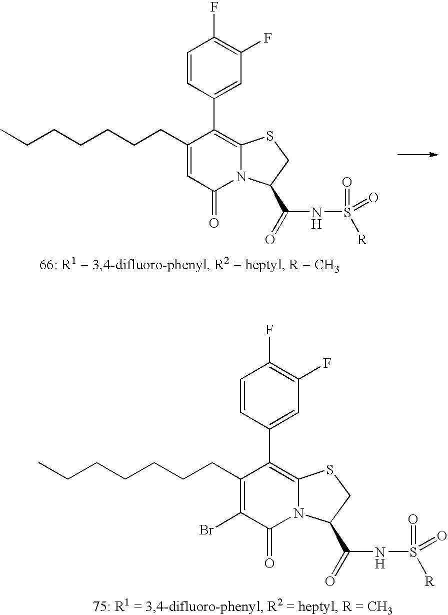

### N-[6-Bromo-8-(3,4-difluoro-phenyl)-7-heptyl-5-oxo-2,3-dihydro-5H-thiazolo[3,2-a]pyridine-3-carbonyl]-methanesulfonamide (75)

Br₂ (1.7 μl, 0.035 mmol) was added drop wise to a stirred solution of 66 (17 mg, 0.035 mmol) in acetic acid (1.5 ml) at rt. After being stirred for 30 min, the reaction mixture was concentrated. Purification with silica gel chromatography (EtOAc→EtOAc:MeOH, 9:1) gave 75 (13.8 mg, 70%). ¹H NMR (400 MHz, CDCl₃) δ 11.05 (bs, 1H), 7.28-7.22 (m, 1H), 7.14-7.05 (m, 1H), 7.03-6.70 (m, 1H), 5.94-5.93 (m, 1H), 3.81-3.76 (m, 2H), 3.33 (s, 3H), 2.47-2.46 (m, 2H), 1.38-1.14 (m, 10H), 0.84 (t, J=7.2 Hz, 3H); ¹³C NMR (100 MHz, CDCl₃) δ 166.1, 158.61, 155.83, 151.73 (splitted), 149.25 (splitted), 147.75, 132.82, 126.63 (splitted), 119.36 (splitted), 118.08 (splitted), 115.58, 111.61, 66.53, 41.66, 34.60, 31.53, 30.44, 29.49, 28.43, 27.79, 22.50, 13.95.

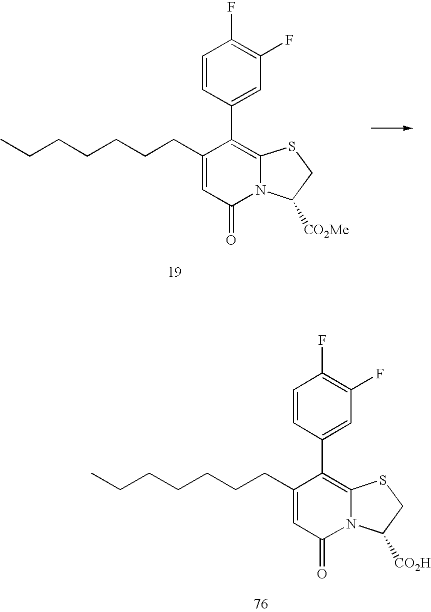

### 8-(3,4-Difluoro-phenyl)-7-heptyl-5-oxo-2,3-dihydro-5H-thiazolo[3,2-a]pyridine-3-carboxylic acid (76)

Compound 76 was prepared as described for 12. ¹³C NMR (100 MHz, CDCl₃) δ 169.15, 162.59, 157.47, 151.54, 149.05, 148.36, 132.76, 126.40, 119.14, 117.92, 116.22, 113.55, 64.89, 33.15, 31.49, 31.12, 29.11, 29.06, 28.73, 22.52, 13.98.

### (3R)-8-(3-(trifluoromethyl)phenyl)-7-hexyl-3,5-dihydro-5-oxo-2H-thiazolo[3,2-a]pyridine-3-carboxylic acid methyl ester (22)

[α]_{D}-116 (c 0.5, CHCl₃); ¹H NMR (400 MHz, CDCl₃) δ 7.52-7.47 (m, 1H), 7.45-7.37 (m, 2H), 7.35-7.29 (m, 1H), 6.07 (s, 1H), 5.51 (dd, J=8.6, 2.1 Hz, 1H), 3.66 (s, 3H), 3.60-3.51 (m, 1H), 3.35-3.28 (m, 1H), 2.12-2.03 (m, 2H), 1.27-1.15 (m, 2H), 1.06-0.89 (m, 6H), 0.63 (t, J=6.9 Hz, 3H); ¹³C NMR (100 MHz, CDCl₃) δ 168.00, 160.76, 155.21, 146.63, 137.07, 133.24 (d, J=27.8 Hz), 130.66 (q, J=32.7 Hz), 128.95, 126.50 (d, J=21.4 Hz), 124.46 (broad, splitted), 123.46 (q, J=272.6 Hz), 114.10, 113.68, 63.18, 52.64, 32.62, 31.18, 30.72, 28.48, 28.19, 21.76, 13.33

### (3R)-8-(3-(trifluoromethyl)phenyl)-7-hexyl-3,5-dihydro-6-nitro-5-oxo-2H-thiazolo[3,2-a]pyridine-3-carboxylic acid methyl ester (30)

By following the procedure described for the preparation of 26 from 21, 22 (776 mg, 1.77 mmol), NaNO₂ (126 mg, 1.83 mmol), 52 ml CH₂Cl₂ and 1.6 ml TFA gave 557 mg (65%) of 30 after purification with silica gel chromatography (heptane:EtOAc, 3:1→1:1). [α]_{D}-204 (c 0.5, CHCl₃); ¹H NMR (400 MHz, CDCl₃) δ 7.72-7.68 (m, 1H), 7.64-7.45 (m, 3H), 5.78-5.74 (m, 1H), 3.83 (s (splitted), 3H), 3.78 (dd, J=8.8, 11.9 Hz, 1H), 3.54-3.48 (m, 1H), 2.36-2.16 (m, 2H), 1.39-1.20 (m, 2H), 1.10-0.98 (m, 4H), 0.98-0.88 (m, 2H), 0.72 (t, J=7.1 Hz, 3H); ¹³C NMR (100 MHz, CDCl₃) δ 167.44 (splitted), 153.39, 151.60 (splitted), 148.38, 138.23 (splitted), 135.63 (splitted), 133.77 (d, J=25.0 Hz), 131.48 (dq, J=32.7, 8.5 Hz), 129.74 (splitted), 127.09 (dq, J=26.2, 3.5 Hz), 125.79 (broad, splitted), 123.56 (q, J=272.5 Hz), 112.57, 64.28 (splitted), 53.52 (splitted), 31.94 (broad), 30.54, 29.46 (broad), 28.89, 28.79, 21.94, 13.65

### (3R)-6-amino-8-(3-(trifluoromethyl)phenyl)-7-hexyl-3,5-dihydro-5-oxo-2H-thiazolo[3,2-a]pyridine-3-carboxylic acid methyl ester (44)

By following the procedure described for the preparation of 42 from 26, 30 (405 mg, 0.84 mmol), Zn dust (328 mg, 5.02 mmol) and 6 ml acetic acid gave 44 as white foam (351 mg, 92%) after purification with silica gel flash chromatography (heptane:EtOAc, 1:2). [α]_{D}-106 (c 0.5, CHCl₃); ¹H NMR (400 MHz, CDCl₃) δ 7.56-7.51 (m, 1H), 7.48-7.40 (m, 2H), 7.39-7.32 (m, 1H), 5.57 (d, J=7.7 Hz, 1H), 4.04 (bs, 2H), 3.71 (s, 3H), 3.59-3.51 (m, 1H), 3.36-3.30 (m, 1H), 2.14-2.05 (m, 2H), 1.35-1.19 (m, 2H), 1.11-0.92 (m, 6H), 0.72-0.64 (t, J=7.0 Hz, 3H); ¹³C NMR (100 MHz, CDCl₃) δ 168.21, 156.36, 138.12 (splitted), 133.29 (d, J=33.8 Hz), 131.15, 130.68, 130.50 (dq, J=31.9, 8.8 Hz), 128.78 (d, J=9.0 Hz), 127.64, 126.63 (d, J=30.2 Hz, broad, splitted), 124.28 (broad, splitted), 123.62 (dq, J=272.2, 3.2 Hz), 115.07, 63.49 (splitted), 52.76 (splitted), 31.51 (broad), 30.75, 28.84, 27.97, 26.62, 21.90, 13.46

### (3R)-6-amino-8-(3-(trifluoromethyl)phenyl)-7-hexyl-3,5-dihydro-5-oxo-2H-thiazolo[3,2-a]pyridine-3-lithium carboxylate (49)

[α]_{D}-93 (c 0.4, DMSO); ¹H NMR (400 MHz, DMSO-d₆) δ 7.74-7.68 (m, 1H), 7.68-7.62 (m, 1H), 7.59-7.51 (m, 1H), 7.46-7.35 (m, 1H), 5.14-5.08 (m, 1H), 4.57 (s, 2H), 3.55-3.45 (m, 1H), 3.47-3.43 (m, 1H), 2.22-2.04 (m, 2H), 1.32-1.16 (m, 2H), 1.11-1.00 (m, 4H), 1.00-0.90 (m, 2H), 0.76-0.68 (t, J=7.1 Hz, 3H); ¹³C NMR (100 MHz, DMSO-d₆) δ 169.01, 156.69, 139.89, 134.88, 132.04, 131.48, 130.09, 129.59 (q, J=30.9 Hz), 127.03 (broad, splitted), 125.05 (broad), 124.60 (q, J=272.5 Hz), 124.56 (broad), 113.33, 67.30, 33.87 (broad), 30.97, 28.97, 27.77 (broad), 26.96, 22.17, 14.25

### Example 1

### (3R)-8-(3,4-Difluoro-phenyl)-7-heptyl-5-oxo-2,3-dihydro-5H-thiazolo[3,2-a]pyridine-3-carboxylic acid

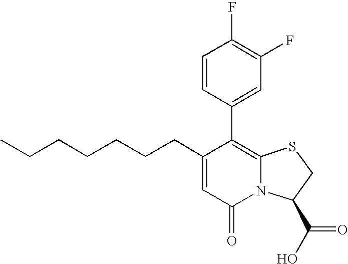

### Example 2

### (3R)—N-[8-(3,4-Difluoro-phenyl)-7-heptyl-5-oxo-2,3-dihydro-5H-thiazolo[3,2-a]pyridine-3-carbonyl]-methanesulfonamide

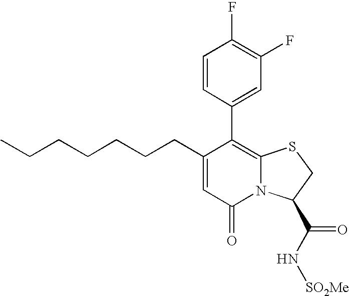

Example 1 (see above) (75 mg, 0.18 mmol) was suspended in CH₂Cl₂ (1 ml) in a microwave vial. To the suspension was added N,N′-carbonyldiimidazole (90 mg, 0.553 mmol) in one portion at room temperature. After 1 hour and 45 minutes, methane sulfonamide (70 mg, 0.736 mmol) was added in one portion at rt. The vial was capped and heated with microwave irradiation (normal absorption) at 80° C. for 7 hours. The solution was diluted with CH₂Cl₂ and washed with 5% aqueous citric acid. The combined aqueous layers were reextracted with CH₂Cl₂ and the combined organic layers were dried, filtrated and concentrated. Purification by silica gel chromatography (EtOAc:MeOH, 9:1) gave Example 2 (58 mg, 65%). ¹H NMR (400 MHz, MeOD-d₄) δ 7.41-7.34 (m, 1H), 7.28-7.24 (m, 1H), 7.12-7.13 (m, 1H), 6.21 (s, 1H), 5.54 (m, 1H), 4.86 (bs, 1H), 3.87-3.81 (m, 1H), 3.54-3.51 (m, 1H), 3.33 (s, 3H), 2.32 (t, J=6.79 Hz, 2H), 1.40-1.36 (m, 2H), 1.25-1.17 (m, 8H), 0.86 (t, J=7.64 Hz, 3H); ¹³C NMR (100 MHz, MeOD-d₄) δ 168.71, 163.47, 158.81, 152.81, 150.72, 150.33, 134.84, 128.40, 120.55, 119.00, 116.39, 113.94, 66.44, 41.39, 34.14, 32.65, 31.97, 30.23, 30.09, 29.76, 23.61, 14.34.

### Example 3

### (2S)-2-[5-(3,4-Difluoro-phenyl)-4-heptyl-2-oxo-2H-pyridin-1-yl]-propionic acid

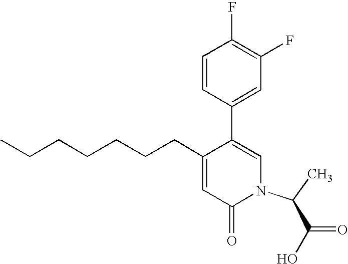

### Example 4

### (3S)-8-(3,4-Difluoro-phenyl)-7-heptyl-5-oxo-2,3-dihydro-5H-thiazolo[3,2-a]pyridine-3-carboxylic acid amide

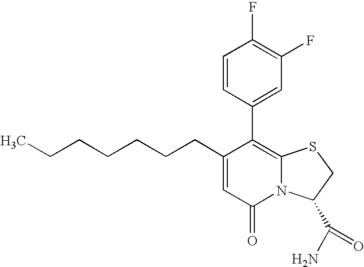

### Example 5

### (3R)-8-(3,4-Difluoro-phenyl)-7-heptyl-3-(1H-tetrazol-5-yl)-2,3-dihydro-thiazolo[3,2-a]pyridin-5-one

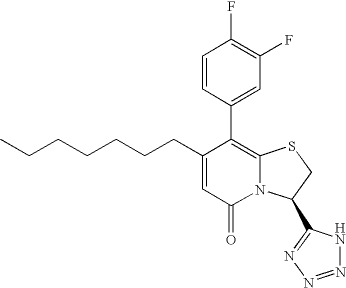

### Example 6

### (3R)-8-(3,4-Difluoro-phenyl)-7-heptyl-5-oxo-2,3-dihydro-5H-thiazolo[3,2-a]pyridine-3,6-dicarboxylic acid 3-methyl ester

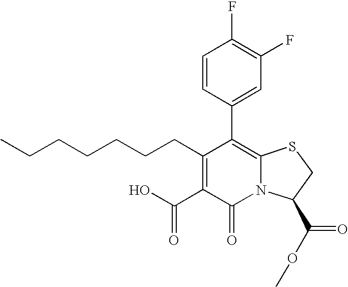

### Example 7

### (3R)-7-Heptyl-5-oxo-8-(3-trifluoromethyl-phenyl)-2,3-dihydro-5H-thiazolo[3,2-a]pyridine-3-carboxylic acid

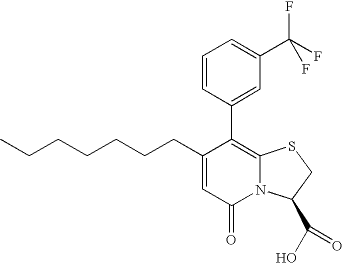

### Example 8

### (3R)—N-[7-Heptyl-5-oxo-8-(3-trifluoromethyl-phenyl)-2,3-dihydro-5H-thiazolo[3,2-a]pyridine-3-carbonyl]-benzenesulfonamide

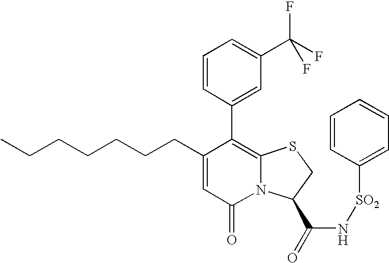

### Example 9

### (3R)-7-Butyl-5-oxo-8-(3-trifluoromethyl-phenyl)-2,3-dihydro-5H-thiazolo[3,2-a]pyridine-3-carboxylic acid

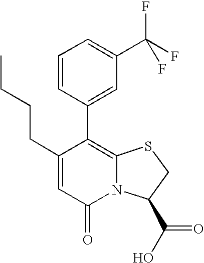

Example 9 was synthesized in the same manner as Example 7.

### Example 10

### (3R)-7-Heptyl-6-nitro-5-oxo-8-(3-trifluoromethyl-phenyl)-2,3-dihydro-5H-thiazolo[3,2-a]pyridine-3-carboxylic acid

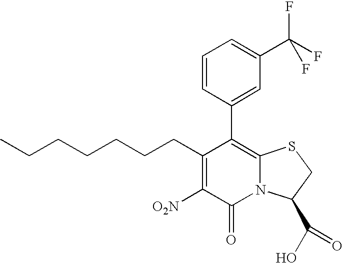

### Example 11

### (3S)-8-(3,4-Difluoro-phenyl)-7-heptyl-5-oxo-2,3-dihydro-5H-thiazolo[3,2-a]pyridine-3-carboxylic acid

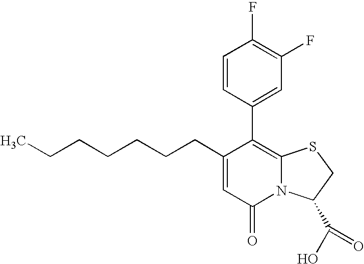

### Example 12

### (3R)-7-Hexyl-6-nitro-5-oxo-8-(3-trifluoromethyl-phenyl)-2,3-dihydro-5H-thiazolo[3,2-a]pyridine-3-carboxylic acid

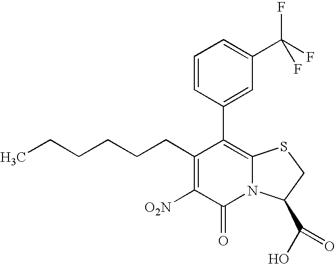

### Example 13

### (3R)-7-(3-methylbutyl)-6-nitro-5-oxo-8-(3-trifluoromethyl-phenyl)-2,3-dihydro-5H-thiazolo[3,2-a]pyridine-3-carboxylic acid

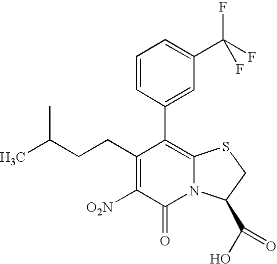

### Example 14

### (3R)-6-Amino-7-hexyl-5-oxo-8-(3-trifluoromethyl-phenyl)-2,3-dihydro-5H-thiazolo[3,2-a]pyridine-3-carboxylic acid

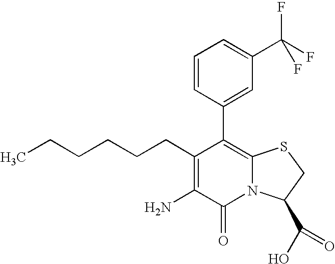

### Example 14 was synthesized starting from compound (49) and for conversion of a lithium salt to the corresponding carboxylic acid, Amberlite® IR-120+ was used (cf. the preparation of compound (12)

### Example 15

### (3R)-7-Butyl-6-nitro-5-oxo-8-(3-trifluoromethyl-phenyl)-2,3-dihydro-5H-thiazolo[3,2-a]pyridine-3-carboxylic acid

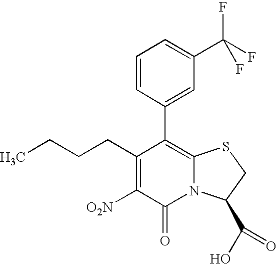

### Example 16

### (3R)-6-Amino-7-(3-methyl-butyl)-5-oxo-8-(3-trifluoromethyl-phenyl)-2,3-dihydro-5H-thiazolo[3,2-a]pyridine-3-carboxylic acid

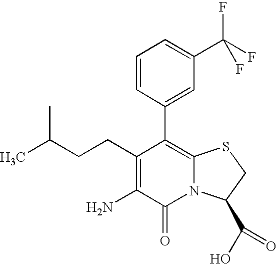

Example 16 was synthesized starting from compound (51) and for conversion of a lithium salt to the corresponding carboxylic acid, Amberlite® IR-120+ was used (cf. the preparation of compound (12).

### Example 17

### (3R)-8-(3,4-Difluoro-phenyl)-7-heptyl-6-nitro-5-oxo-2,3-dihydro-5H-thiazolo[3,2-a]pyridine-3-carboxylic acid

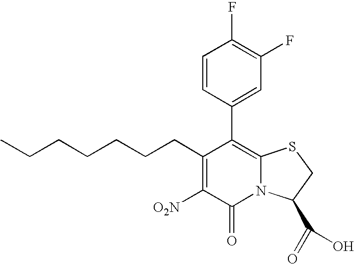

### Example 18

### (3R)-7-octyl-5-oxo-8-[3-(trifluoromethyl)phenyl]-2,3-dihydro-5H-[1,3]thiazolo[3,2-a]pyridine-3-carboxylic acid

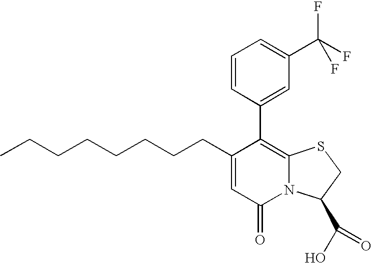

Example 18 was prepared in three steps a)-c):

### a) Preparation of 5-(1-hydroxynonylidene)-2,2-dimethyl-[1,3]dioxane-4,6-dione

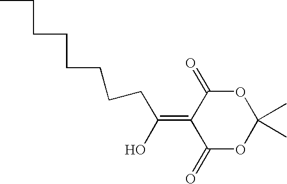

Prepared according to previously published procedure (Emtenas, H.; Taflin, C.; Almqvist, F. Mol. Diversity, 2003, 7, 165-169). Starting from nonanoic acid (3 gm, 18.9 mmol) gave the title compound in step a) as oil (5.2 gm, 97%). ¹H NMR (400 MHz, CDCl₃) δ 3.07 (t, J=7.7 Hz, 2H), 1.73 (s, 6H), 1.42-1.28 (m, 12H), 0.88 (t, J=7.1 Hz, 3H). ¹³C NMR (100 MHz, CDCl₃) δ 198.21, 104.63, 91.15, 35.62, 31.67, 29.26, 29.07, 28.97, 26.67 (2C), 26.04, 22.51, 13.94.

### b) Preparation of (3R)-7-octyl-5-oxo-8-(3-trifluoromethylphenyl)-2,3-dihydro-5H-thiazolo[3,2-a]pyridine-3-carboxylic acid methyl ester

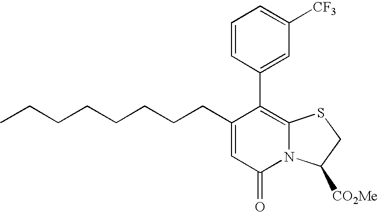

Prepared as described for 9 (Method 2); Starting from thiazoline derivative 5 (500 mg, 1.65 mmol) gave the title compound in step b) as oil (673 mg, 87%). ¹H NMR (400 MHz, CDCl₃) δ 7.61 (d, J=8 Hz, 1H), 7.54-7.5 (m, 2H), 7.41 (d, J=8 Hz, 1H), 6.21 (s, 1H), 5.62 (dd, J=2.4, 8.8 Hz, 1H), 3.8 (s, 3H), 3.64 (t, J=9.2 Hz, 1H), 3.43 (d, J=11.2 Hz, 1H), 2.18 (t, J=7.6 Hz, 2H), 1.34-1.29 (m, 2H), 1.21-1.10 (m, 10H), 0.79 (t, J=7.2 Hz, 3H). ¹³C NMR (100 MHz, CDCl₃) δ 168.3, 161.2, 155.7, 146.7, 137.3, 133.7-133.3 (m, 1C), 131.3-131.0 (m, 1C), 129.3, 127.0-126.8 (m, 1C), 125.1-124.9 (m, 1C), 122.4, 114.7, 114.2, 63.5, 53.2, 33.0, 31.6 (2C), 28.9 (4C), 22.4, 13.9.

### c) Preparation of (3R)-7-octyl-5-oxo-8-(3-trifluoromethylphenyl)-2,3-dihydro-5H-thiazolo[3,2-a]pyridine-3-carboxylic acid

Prepared as described for 12; Starting from the title compound in step b) (22 mg, 0.047 mmol) gave the title compound in step c) as solid (20 mg, quant.). ¹H NMR (400 MHz, CDCl₃) δ 10.26 (bs, 1H), 7.66 (d, J=7.6 Hz, 1H), 7.55-7.51 (m, 2H), 7.43 (d, J=6.8 Hz, 1H), 6.39 (d, J=6.8 Hz, 1H), 5.78-5.76 (m, 1H), 3.73-3.66 (m, 2H), 2.23 (t, J=7.6, 2H), 1.36-1.35 (m, 2H), 1.25-1.11 (m, 10H), 0.83 (t, J=7.2 Hz, 3H). ¹³C NMR (100 MHz, CDCl₃) δ 168.69, 162.59, 157.44, 148.22, 136.88, 133.61-133.16 (m, 1C), 129.53-129.40 (m, 1C), 127.09-127.01 (m, 1C), 125.25, 116.88, 113.78 (2C), 64.65, 33.18, 31.66, 31.24, 29.08 (2C), 28.96, 28.90, 22.51, 13.98.

### Example 19

### (3R)-7-heptyl-5-oxo-8-(2-thienyl)-2,3-dihydro-5H-[1,3]thiazolo[3,2-a]pyridine-3-carboxylic acid

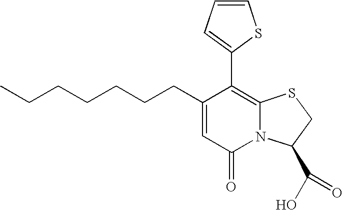

Example 19 was prepared in two steps a)-b):

### a) Preparation of (3R)-7-Heptyl-5-oxo-8-thiophen-2-yl-2,3-dihydro-5H-thiazolo[3,2-a]pyridine-3-carboxylic acid methyl ester

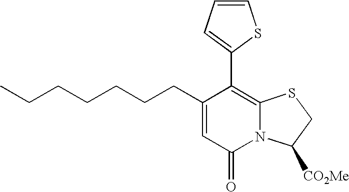

Prepared as described for 9 (Method 2); Starting from thiazoline derivative (made using conditions described for 5, where R¹=Thiophene) (140 mg, 0.58 mmol) gave the title compound in step a) as oil (195 mg, 86%). ¹H NMR (400 MHz, CDCl₃) δ 7.37 (d, J=5.2 Hz, 1H), 7.05 (t, J=4.8, 1H), 6.94 (d, J=undefined, 1H), 6.19 (s, 1H), 5.62 (d, J=8.8 Hz, 1H), 3.81 (s, 3H), 3.66-3.61 (m, 1H), 3.43 (d, J=11.6 Hz, 1H), 2.31 (t, J=7.2 Hz, 2H), 1.42-1.41 (m, 2H), 1.23-1.17 (m, 8H), 0.83 (t, J=7.2 Hz, 3H). ¹³C NMR (100 MHz, CDCl₃) δ 168.38, 161.22, 157.12, 148.93, 136.78, 128.79, 127.05, 126.91, 113.79, 108.48, 63.70, 53.19, 33.27, 31.46 (2C), 29.13, 29.03, 28.74, 22.45, 13.95.

### b) Preparation of (3R)-7-heptyl-5-oxo-8-thiophen-2-yl-2,3-dihydro-5H-thiazolo[3,2-a]pyridine-3-carboxylic acid

Prepared as described for 12; Starting from the title compound in step a) (48.7 mg, 0.124 mmol) gave the title compound in step b) as solid (46 mg, quant.). ¹H NMR (400 MHz, DMSO-d₆) δ 7.65 (d, J=5.2 Hz, 1H), 7.13-7.11 (dd, J=3.6, 5.2, 1H), 7.01 (d, J=3.6, 1H), 6.05 (s, 1H), 5.45 (dd, J=1.3, 8.9 Hz, 1H), 3.81 (dd, J=9.2, 11.6 Hz, 1H), 3.47-3.44 (m, 1H), 2.32-2.26 (m, 2H), 1.34-1.32 (m, 2H), 1.22-1.13 (m, 8H), 0.81 (t, J=7.2 Hz, 3H). ¹³C NMR (100 MHz, CDCl₃) δ 168.67, 162.59, 158.98, 150.46, 136.15, 129.02, 127.24 (2C), 113.35, 110.89, 64.88, 33.43, 31.50, 30.96, 29.36, 29.15, 28.75, 22.51, 13.99.

### Example 20

### (3R)-7-heptyl-8-(1H-indol-3-yl)-5-oxo-2,3-dihydro-5H-[1,3]thiazolo[3,2-a]pyridine-3-carboxylic acid

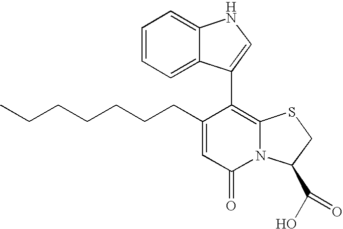

Example 20 was prepared in two steps a)-b):

### a) Preparation of (3R)-7-heptyl-5-oxo-8-[1-(3-oxo-decanoyl)-1H-indol-3-yl]-2,3-dihydro-5H-thiazolo[3,2-a]pyridine-3-carboxylic acid methyl ester

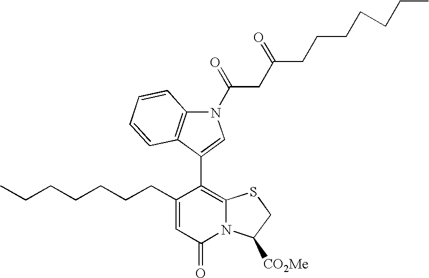

Prepared as described for 9 (Method 2); Starting from thiazoline (made using conditions described for 5, where R¹=1H-Indole) (119.2 mg, 0.434 mmol) gave the title compound in step a) as oil (203 mg, 79%). ¹H NMR (400 MHz, CDCl₃) δ 8.55 (t, J=8.4 Hz, 1H), 7.49-7.29 (m, 4H), 6.32-6.31 (m, 1H), 5.73-5.71 (m, 1H), 4.06 (d, J=4.4 Hz, 1H), 3.87 (s, 3H), 3.71-3.65 (m, 1H), 3.48-3.44 (m, 1H), 2.68 (t, J=7.2 Hz, 1H), 2.39-2.29 (m, 3H), 1.72-1.65 (m, 2H), 1.43-1.12 (m, 19H), 0.91-0.84 (m, 6H). ¹³C NMR (100 MHz, CDCl₃) δ 202.44, 183.84, 170.20, 168.61, 168.51, 168.47, 164.82, 161.57, 157.52, 157.34, 135.65, 129.84, 126.05, 125.53, 124.46, 124.43, 124.08, 123.73, 123.18, 119.83, 119.76, 119.71, 119.64, 116.95, 116.93, 116.82, 116.77, 113.99, 113.93, 113.91, 113.78, 88.80, 63.89, 63.60, 53.28, 53.29, 51.73, 51.41, 43.36, 43.25, 36.15, 33.30, 33.27, 33.26, 33.22, 31.64, 31.58, 31.49, 31.46, 29.11, 28.96, 28.92, 28.81, 28.77, 26.49, 26.48, 23.46, 23.45, 22.57, 22.53, 22.50, 22.49, 14.03, 14.01, 13.98, 13.97.

### b) Preparation of (3R)-7-heptyl-8-(1H-indol-3-yl)-5-oxo-2,3-dihydro-5H-thiazolo[3,2-a]pyridine-3-carboxylic acid

Prepared as described for 12; Starting from the title compound in step a) (55.3 mg, 0.093 mmol) gave the title compound in step b) (purified by passing through small silica gel column (EtOAc 100%)) as solid (38 mg, quant.). ¹H NMR (400 MHz, CDCl₃) δ 8.63 (s, 1H), 7.70 (bs, 1H), 7.44 (d, J=7.2 Hz, 1H), 7.34-7.24 (m, 2H), 7.16-7.11 (m, 2H), 6.42 (s, 1H), 5.76 (d, J=6.8 Hz, 1H), 3.74 (d, J=7.6 Hz, 1H), 3.59-3.52 (m, 1H), 2.32 (t, J=7.2 Hz, 2H), 1.36-1.35 (m, 2H), 1.06-1.05 (m, 8H), 0.80-0.77 (m, 3H). ¹³C NMR (100 MHz, CDCl₃) δ 168.65, 163.18, 160.43, 160.26, 149.86, 149.71, 135.96, 135.80, 126.57, 125.98, 124.70, 123.47, 122.63, 122.61, 120.28, 120.22, 119.50, 119.15, 113.72, 112.95, 112.86, 111.59, 111.54, 111.39, 111.30, 65.31, 33.39, 33.30, 31.41, 31.40, 29.21, 29.03, 28.96, 28.95, 28.70, 28.69, 22.44, 13.93.

### Example 21

### (3R)-7-[3-(2,4-dichloro-phenoxy)-propyl]-5-oxo-8-phenyl-2,3-dihydro-51H-thiazolo[3,2-a]pyridine-3-carboxylic acid

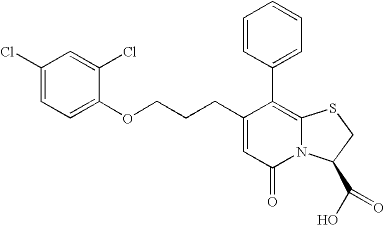

The corresponding methyl ester was prepared as described for 9 (Method 1); ¹H NMR (400 MHz, CDCl₃) δ 7.39-7.35 (m, 3H), 7.30 (d, J=2.45 Hz, 1H), 7.23-7.21 (m, 2H), 7.10 (dd, J=8.79, 2.60 Hz, 1H), 6.67 (d, J=8.8 Hz, 1H), 6.28 (s, 1H), 5.65 (dd, J=8.6, 2.4 Hz, 1H), 3.86 (t, J=6.0 Hz, 2H), 3.82 (s, 3H), 3.67-3.62 (m, 1H), 3.46-3.42 (m, 1H), 2.52 (t, J=7.74 Hz, 2H), 1.90-1.84 (m, 2H); ¹³C NMR (100 MHz, CDCl₃) δ 168.46, 161.33, 154.91, 152.87, 146.83, 136.13, 130.00, 129.82, 129.60, 128.83, 128.26, 127.37, 127.35, 125.53, 123.63, 116.31, 114.08, 113.78, 67.87, 63.54, 53.27, 31.59, 29.57, 28.18.

Example 21 was prepared as described for 12 starting from the corresponding methyl ester of Example 21; ¹H NMR (400 MHz, MeOD-d4) δ 7.40-7.35 (m, 3H), 7.34-7.33 (m, 1H), 7.28-7.17 (m, 3H), 6.88 (d, J=8.9 Hz, 1H), 6.26 (s, 1H), 5.65 (dd, J=8.84, 1.72 Hz, 1H), 3.93-3.89 (m, 2H), 3.84-3.78 (m, 1H), 3.56-3.52 (m, 1H), 2.60-2.56 (m, 2H), 1.88-1.81 (m, 2H); ¹³C NMR (100 MHz, MeOD-d4) δ 169.69, 162.36, 156.21, 153.16, 148.75, 136.29, 129.94, 129.70, 129.30, 128.68, 128.12, 127.47, 125.17, 123.30, 117.31, 114.05, 112.98, 67.68, 64.11, 31.39, 29.5, 28.29.

The yield of Example 21 is 66% starting from cysteine.

### Example 22

### (2S,3R)-8-(3,4-difluorophenyl)-7-heptyl-2-methoxy-5-oxo-2,3-dihydro-5H-[1,3]thiazolo[3,2-a]pyridine-3-carboxylic acid

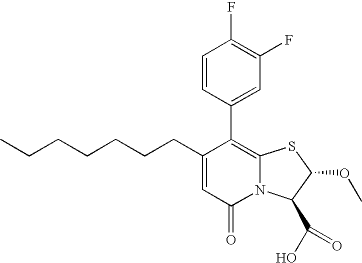

### Example 23

### 8-(3,4-difluorophenyl)-7-heptyl-5-oxo-5H-[1,3]thiazolo[3,2-a]pyridine-3-carboxylic acid

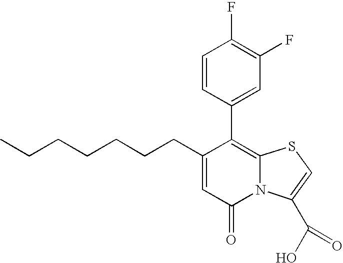

### Example 24

### (2R,3R)-8-(3,4-difluorophenyl)-7-heptyl-5-oxo-2-phenyl-2,3-dihydro-5H-[1,3]thiazolo[3,2-a]pyridine-3-carboxylic acid

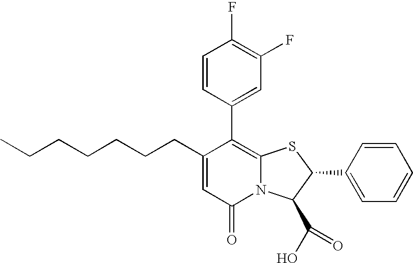

### Example 25

### (2R,3R)-8-(3,4-difluorophenyl)-7-heptyl-2-methyl-5-oxo-2,3-dihydro-5H-[1,3]thiazolo[3,2-a]pyridine-3-carboxylic acid

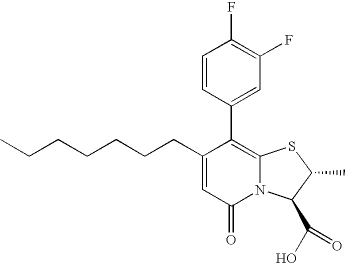

The procedures from 10 to 77 and 77 to Example 22, 78 and 79 on other substrates is published in Chorell, E.; Das, P.; Almqvist, F. J. Org. Chem. 2007, 72, 4917-4924.

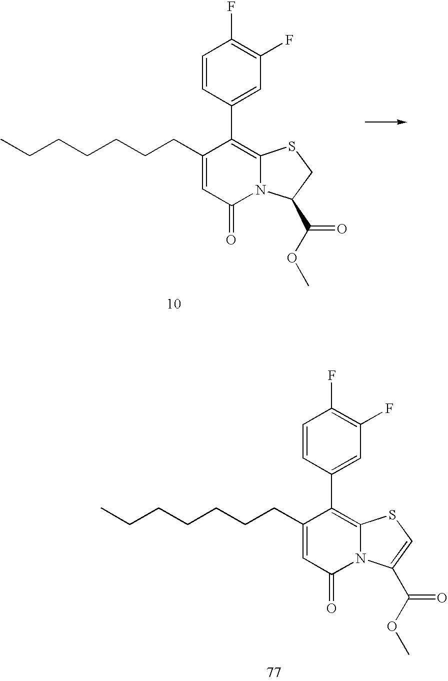

### 8-(3,4-Difluoro-phenyl)-7-heptyl-1-ylmethyl-5-oxo-5H-thiazolo[3,2-a]pyridine-3-carboxylic acid methyl ester (77)

NaH (28 mg, 1.185 mmol, washed with n-pentane) was added to 1 (200 mg, 0.474 mmol) dissolved in 3.25 mL of dry THF at 0° C. while stirring. After 10 min BrCCl₃ (0.047 mL, 0.474 mmol) was added and the mixture was allowed to attain rt and stirred for an additional 10 min followed by the addition of dry NaOMe (19 mg, 0.356 mmol). After approximately 1.5 h of stirring at rt the reaction was quenched by dropwise addition of 2% aqueous KHSO₄ (The reaction was carefully monitored with TLC and one could also observe a color change from pale yellow to yellow-orange upon completion). The mixture was acidified and then extracted three times by EtOAc. The combined organic layers was washed with brine, dried (Na₂SO₄), filtered and concentrated. Purification by silica gel chromatography (heptane/EtOAc, 3/2) gave the title compound as an oil (121 mg, 61%): ¹H NMR (400 MHz, CDCl₃) δ 7.32-7.23 (m, 1H), 7.14-7.07 (m, 1H), 7.05-6.99 (m, 2H), 6.29 (s, 1H), 3.95 (s, 3H), 2.33 (t, J=7.42 Hz, 2H), 1.44-1.33 (m, 2H), 1.26-1.09 (m, 8H), 0.82 (t, J=6.95 Hz, 3H); ¹³C NMR (100 MHz, CDCl₃) δ 160.8, 159.1, 153.3, 151.9, 149.4, 147.4, 132.8, 131.7, 126.7, 119.4, 118.6, 113.8, 113.2, 110.5, 53.5, 32.2, 31.6, 29.7, 29.1, 28.9, 22.6, 14.1.

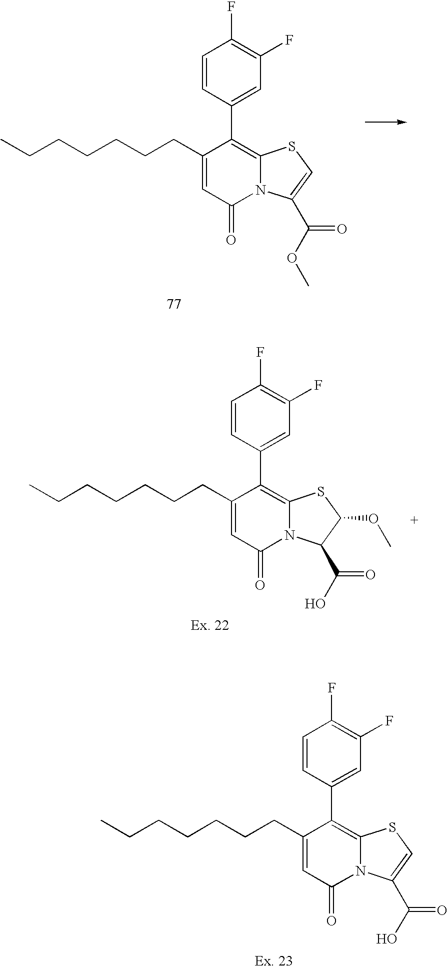

n-BuLi (1.6 M, 0.143 mmol) was added dropwise to 0.2 mL of MeOH in 0.5 mL of THF at −78° C. After stirring for 5 min the solution was allowed to attain rt followed by the addition of 77 (20 mg, 0.048 mmol) dissolved in 0.6 mL of THF. The solution was stirred for 4 h at rt before being concentrated. Purification by silica gel chromatography (DCM/MeOH/AcOH, 95/4/1) gave Example 22 (8.3 mg, 40% yield) and Example 23 (8.6 mg, 44%) as oils.

Example 22: ¹H NMR (400 MHz, CDCl₃) δ 7.30-7.19 (m, 1H), 7.14-6.91 (m, 2H), 6.41 (s, 1H), 5.78 (s, 1H), 5.68 (s, 1H), 3.38 (s, 3H), 2.27 (t, J=7.82 Hz, 2H), 1.44-1.33 (m, 2H), 1.29-1.11 (m, 8H), 0.85 (t, J=6.95 Hz, 3H). MS⁺ calcd for [M+H]⁺ C₂₂H₂₆F₂NO₄S 438.1545, obsd 438.14.

Example 23: ¹H NMR (400 MHz, CD₃OD) δ 7.44-7.19 (m, 2H), 7.17-7.05 (m, 1H), 6.27 (s, 1H), 5.62 (s, 1H), 2.39-2.29 (m, 2H), 1.44-1.34 (m, 2H), 1.31-1.12 (m, 8H), 0.85 (t, J=7.0 Hz, 3H). MS calcd for [M+H]C₂₁H₂₂F₂NO₃S 406.1283, obsd 406.10.

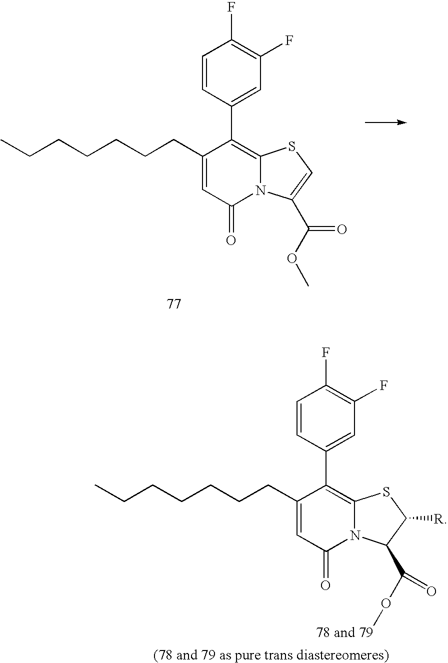

### General Procedure for the Preparation of 78 and 79.

Preparation of cuprate: A solution of RLi (R=Ph for 78 (2.0 M in dibutyl ether, 0.5 mL, 1.0 mmol) and R=Me for 79 (1.6 M in diethyl ether, 0.625 mL, 1.0 mmol)) was added dropwise to CuCN (45 mg, 0.50 mmol) in 2 mL of THF at −78° C. Stirred at −78° C. for 10 min before warmed to 0° C. and stirred there for 10 min before the clear solution was again cooled to −78° C.

1.5 equiv cuprate (Ph₂CuCNLi₂: 0.2 M for 78, Me₂CuCNLi₂: 0.19 M for 79) was added to 77 in 2 mL of THF at −78° C. After stirring for 20 min the reaction was quenched with aqueous saturated NH₄Cl and the solution were extracted three times with DCM. The combined organic layers was dried (Na₂SO₄), filtered and concentrated; the residue was purified by column chromatography (heptane/EtOAc, 3/2) giving 78 or 79.

77 (30 mg, 0.072 mmol) gave 78 as an oil (26.0 mg, 73%): ¹H NMR (400 MHz, CDCl₃) δ 7.38-7.25 (m, 5H), 7.25-7.15 (m, 1H), 7.15-7.05 (m, 1H), 7.04-6.96 (m, 1H), 6.27 (s, 1H), 5.65 (d, J=2.83 Hz, 1H), 4.96 (d, J=2.97 Hz, 1H), 3.86 (s, 3H), 2.26 (t, J=7.54 Hz, 2H), 1.45-1.35 (m, 2H), 1.29-1.12 (m, 8H), 0.85 (t, J=6.90 Hz, 3H); ¹³C NMR (100 MHz, CDCl₃) δ 168.1, 161.2, 156.0, 151.5, 149.0, 146.5, 138.5, 133.2, 129.3, 128.9, 126.7, 126.5, 119.3, 117.8, 114.4, 113.9, 70.8, 53.4, 50.9, 33.2, 31.5, 29.1, 28.9, 28.8, 22.5, 14.0.

77 (30 mg, 0.072 mmol) gave 79 as an oil (14 mg, 45%): ¹H NMR (400 MHz, CDCl₃) δ 7.25-7.15 (m, 1H), 7.11-7.03 (m, 1H), 7.00-6.94 (m, 2H), 6.24 (s, 1H), 5.29 (s, 1H), 3.97-3.89 (m, 1H), 3.82 (s, 3H), 2.23 (t, J=7.52 Hz, 2H), 1.55 (d, J=6.92 Hz, 3H) 1.43-1.33 (m, 2H), 1.28-1.11 (m, 8H), 0.84 (t, J=6.93 Hz, 3H); ¹³C NMR (100 MHz, CDCl₃) δ 168.2, 161.6, 155.9, 151.5, 149.0, 146.5, 133.3, 126.6, 119.3, 117.7, 114.3, 114.1, 70.2, 53.3, 43.3, 33.2, 31.5, 29.1, 28.9, 28.8, 22.9, 22.5, 14.0.

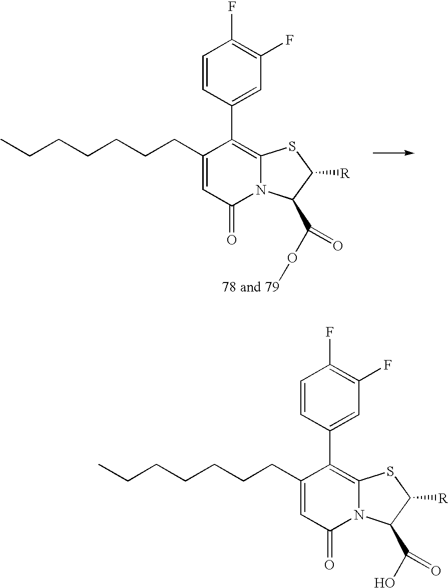

### Example 24 and 25

(Example 24 and 25 as Pure Trans Diastereomeres.)

Compound 78 (0.025 mg, 0.050 mmol) was dissolved in 1.75 mL THF/MeOH (1:4) and 0.1 M LiOH (aq.) (0.5 mL, 0.050 mmol) was added dropwise at rt. The solution was stirred overnight and was then concentrated twice from MeOH before being dissolved in MeOH and treated with Amberlite® IR120+ ion-exchange resin to yield Example 24 (quant.).

Compound 79 (0.02 mg, 0.048 mmol) was dissolved in 1.75 mL THF/MeOH (1:4) and 0.1 M LiOH (aq.) (0.48 mL, 0.05 mmol) was added dropwise at rt. The solution was stirred overnight and was then concentrated twice from MeOH before being dissolved in MeOH and treated with Amberlite® IR120+ ion-exchange resin to yield Example 25 (quant.). ¹H NMR (400 MHz, CD₃OD) δ 7.41-7.16 (m, 2H), 7.15-7.02 (m, 1H), 6.23 (s, 1H), 5.33 (s, 1H), 4.08 (q, J=6.94 Hz, 1H), 2.36-2.28 (m, 2H), 1.54 (d, J=6.73 Hz, 3H), 1.44-1.33 (m, 2H), 1.30-1.12 (m, 8H), 0.85 (t, J=6.96 Hz, 3H).

### Plasma Clot Lysis Assay for Testing of PAI-1 Inhibitors

### Materials

Two-chain tPA

CaCl₂, p.a. Stock solution 0.1 M in water.

Citrate, 0.13 M

Recombinant human PAI-1

PAI-1 inhibitors dissolved in 100% DMSO.

### Experimental Procedures—Clot Lysis in Platelet-Poor Plasma

Blood from healthy fat-fasting volunteers was collected into 0.13 M trisodium citrate, 9 parts blood to 1 part anticoagulant. The tubes were centrifuged at 2000×g, 20 min, at RT. The supernatant, ie, the platelet poor plasma was pooled, aliquoted and frozen at −85° C. until used. At the day of experiment the plasma was thawed in a water bath and temperated to 37° C. All other constituents, except t-PA, were prewarmed to 37° C. To each well on a microtiter plate, 25 μL CaCl₂, 25 μL PAI-1 or 25 μL PAI-1 vehicle, 20 μL saline and 5 μL drug or 5 μL vehicle (100% DMSO) were added. Plasma was mixed with cold t-PA solution in the relation 4 parts plasma with 1 part t-PA, just before adding 125 μL of this mixture to each well. The final concentration was 12.5 mM for CaCl₂, 10-13 ng/mL for PAI-1, 34 ng/mL for t-PA, and the compounds were tested at a final concentration range 0.1 nM to 0.25 mM. The final plasma concentration was 50%, as a result of the different additives. The plate, covered with a plastic lid, was placed in a Microplate reader (Molecular Devices, US) and gently shaken. The change in turbidity was immediately monitored as a change in absorbance at 405 nm at 37° C. Data points were collected at intervals of 2 min, during a period of 10 h. After finished reading, the absorbance data were transformed into files containing the time and absorbance values for each well. The clot longevity, ie, the time the clot exists, was determined as the time between clot formation, ie, positive Vₘₐₓ, and clot lysis, ie, negative Vₘₐₓ. The effect of the drugs, ie, shortening of the longevity for the clot, was expressed as the concentration at which a halving of the clot lysis time was reached, the IC₅₀ (pIC₅₀=−log IC₅₀). Control clot lysis time, set to 100%, was determined in the presence of PAI-1, and the maximal effect, ie, the shortest lysis time that can be reached under defined conditions in this system, in the absence of PAI-1 was set to 0%.

Compounds having a pIC₅₀ higher than 4 were considered as being active. The following pIC₅₀ values are presented:

| Example | pIC₅₀ |
|---|---|
| 1 | 4.8 |
| 2 | 5.0 |
| 3 | 4.9 |
| 4 | 4.3 |
| 5 | 4.6 |
| 6 | 4.7 |
| 7 | 4.9 |
| 8 | 5.6 |
| 9 | 4.4 |
| 10 | 5.4 |
| 11 | 5.0 |
| 12 | 4.9 |
| 13 | 4.9 |
| 14 | 4.9 |
| 15 | 4.4 |
| 16 | 4.1 |
| 17 | 4.7 |
| 18 | 5.4 |
| 19 | 5.0 |
| 20 | 4.9 |
| 21 | 4.9 |
| 22 | 5.0 |
| 23 | 5.0 |
| 24 | 5.3 |
| 25 | 5.4 |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

What is claimed is:

1. A compound of formula (I), (II), or (III): 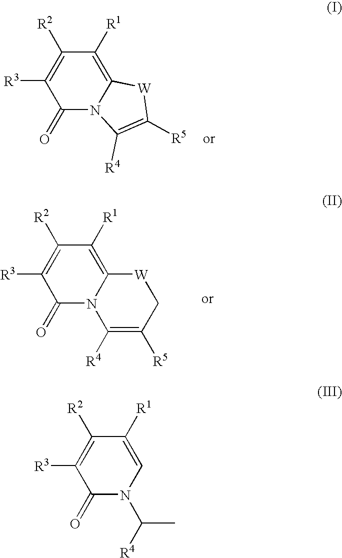
or a pharmaceutically acceptable salt or enantiomer thereof wherein:
W is selected from S, SO, SO₂, O, P, PO, PO₂, and CH₂;
R¹ is (CH₂)ₘD wherein m is a natural number being 0, 1, 2, 3, 4, or 5 and D is selected from hydrogen, alkyl, alkenyl, alkynyl, unsubstituted or substituted aryl, unsubstituted or substituted heteroaryl, substituted alkyl, substituted alkenyl, substituted alkynyl, and unsubstituted or substituted cycloalkyl;
R² is selected from C₂-C₄-alkyl; unsubstituted or substituted isopentyl; unsubstituted or substituted C₆-C₁₀-alkyl; unsubstituted or substituted cycloalkylmethyl; unsubstituted or substituted (CH₂)ₘ-cycloalkyl, unsubstituted or substituted (CH₂)ₘ-aryl, wherein m is a natural number being 2, 3, 4, or 5; and (CH₂)ₙA wherein n is a natural number being 0, 1, 2, 3, 4, or 5 and A is selected from alkenyl, alkynyl, aryloxy, heteroaryl, substituted alkenyl, substituted alkynyl, substituted aryloxy, and substituted heteroaryl;
R³ is selected from hydrogen, halogen, nitro, hydroxyalkyl, carboxy, and —NHR⁰ wherein R⁰ is selected from hydrogen, alkylsulfonyl, acyl, acyl substituted by acylamido and hydroxyalkyl;
R⁴ is selected from CO₂Y, B(OY)₂, CHO, CH₂OY, CH(CO₂Y)₂, PO(OY)₂ wherein Y is selected from hydrogen, alkyl, alkenyl, alkynyl, aryl, heteroaryl, substituted alkyl, substituted alkenyl, substituted alkynyl, cycloalkyl, substituted aryl or substituted heteroaryl; tetrazolyl; and CONHZ wherein Z is selected from hydrogen, hydroxy, alkyl, alkylsulfonyl, arylsulfonyl, and cyanoalkyl; and
R⁵ is selected from hydrogen, alkyl, alkoxy, unsubstituted or substituted aryl, and unsubstituted or substituted heteroaryl.

2. The compound according to claim 1 of formula (I) or (III) wherein:
W is S or SO₂;
R¹ is (CH₂)ₘD wherein m is 0 and D is selected from unsubstituted or substituted cycloalkyl, unsubstituted or substituted aryl, and unsubstituted or substituted heteroaryl;
R² is selected from C₂-C₄-alkyl; isopentyl; C₆-C₁₀-alkyl; (CH₂)ₘ-aryl wherein m is a natural number being 2, 3, 4, or 5; and (CH₂)ₙA wherein n is a natural number being 0, 1, 2, 3, 4, or 5, and A is substituted aryloxy;
R³ is selected from hydrogen, halogen, nitro, hydroxyalkyl, carboxy, and —NHR⁰ wherein R⁰ is selected from hydrogen, alkylsulfonyl, acyl, acyl substituted by acylamido and hydroxyalkyl;
R⁴ is selected from CO₂Y wherein Y is selected from hydrogen or alkyl; tetrazolyl; and CONHZ wherein Z is selected from hydrogen, hydroxy, alkyl, alkylsulfonyl, arylsulfonyl, and cyanoalkyl; and
R⁵ is selected from hydrogen, alkyl, alkoxy, unsubstituted or substituted aryl, and unsubstituted or substituted heteroaryl.

3. The compound according to claim 1 wherein R⁵ is selected from hydrogen, alkyl, alkoxy, and unsubstituted or substituted aryl.

4. The compound according to claim 1 of formula (I) or (III) wherein:
W is S;
R¹ is (CH₂)ₘD wherein m is 0 and D is cycloalkyl, or unsubstituted or substituted aryl;
R² is selected from C₂-C₄-alkyl; isopentyl; C₆-C₁₀-alkyl; and (CH₂)ₙA wherein n is 3 and A is 2,4-dichlorophenoxy;
R³ is selected from hydrogen, halogen, nitro, hydroxyalkyl, carboxy, and —NHR⁰ wherein R⁰ is selected from hydrogen, alkylsulfonyl, acyl, acyl substituted by acylamido and hydroxyalkyl;
R⁴ is CO₂Y wherein Y is selected from hydrogen; tetrazolyl; and CONHZ wherein Z is alkylsulfonyl or arylsulfonyl; and
R⁵ is selected from hydrogen, methyl, methoxy, and phenyl.

5. The compound according to claim 1 wherein aryl is C₆₋₁₅ aryl, aryloxy is C₆₋₁₅ aryloxy, alkenyl is C₁₋₁₅ alkenyl, alkynyl is C₁₋₁₅ alkynyl, cycloalkyl is C₃₋₆ alkyl, and heteroaryl is C₅₋₁₅ heteroaryl.

6. The compound according to claim 1 wherein substituted aryl is aryl substituted by one or more fluoro.

7. The compound according to claim 1 wherein substituted aryl is aryl substituted by one or more trifluoromethyl.

8. A compound according to claim 1 of formula (I), (II), or (III) wherein the stereochemical configuration around the carbon which is covalently bound to R⁴ is (R).

9. A compound according to claim 1 of formula (I), (II), or (III) wherein the stereochemical configuration around the carbon which is covalently bound to R⁴ is (S).

10. The compound according to claim 1 which is selected from:
(3R)-8-(3,4-Difluoro-phenyl)-7-heptyl-5-oxo-2,3-dihydro-5H-thiazolo[3,2-a]pyridine-3-carboxylic acid;
(3R)—N-[8-(3,4-Difluoro-phenyl)-7-heptyl-5-oxo-2,3-dihydro-5H-thiazolo[3,2-a]pyridine-3-carbonyl]-methanesulfonamide;
(2S)-2-[5-(3,4-Difluoro-phenyl)-4-heptyl-2-oxo-2H-pyridin-1-yl]-propionic acid;
(3S)-8-(3,4-Difluoro-phenyl)-7-heptyl-5-oxo-2,3-dihydro-5H-thiazolo[3,2-a]pyridine-3-carboxylic acid amide;
(3R)-8-(3,4-Difluoro-phenyl)-7-heptyl-3-(1H-tetrazol-5-yl)-2,3-dihydro-thiazolo[3,2-a]pyridin-5-one;
(3R)-8-(3,4-Difluoro-phenyl)-7-heptyl-5-oxo-2,3-dihydro-5H-thiazolo[3,2-a]pyridine-3,6-dicarboxylic acid 3-methyl ester;
(3R)-7-Heptyl-5-oxo-8-(3-trifluoromethyl-phenyl)-2,3-dihydro-5H-thiazolo[3,2-a]pyridine-3-carboxylic acid;
(3R)—N-[7-Heptyl-5-oxo-8-(3-trifluoromethyl-phenyl)-2,3-dihydro-5H-thiazolo[3,2-a]pyridine-3-carbonyl]-benzenesulfonamide;
(3R)-7-Butyl-5-oxo-8-(3-trifluoromethyl-phenyl)-2,3-dihydro-5H-thiazolo[3,2-a]pyridine-3-carboxylic acid;
(3R)-7-Heptyl-6-nitro-5-oxo-8-(3-trifluoromethyl-phenyl)-2,3-dihydro-5H-thiazolo[3,2-a]pyridine-3-carboxylic acid;
(3S)-8-(3,4-Difluoro-phenyl)-7-heptyl-5-oxo-2,3-dihydro-5H-thiazolo[3,2-a]pyridine-3-carboxylic acid;
(3R)-7-Hexyl-6-nitro-5-oxo-8-(3-trifluoromethyl-phenyl)-2,3-dihydro-5H-thiazolo[3,2-a]pyridine-3-carboxylic acid;
(3R)-7-(3-methylbutyl)-6-nitro-5-oxo-8-(3-trifluoromethyl-phenyl)-2,3-dihydro-5H-thiazolo[3,2-a]pyridine-3-carboxylic acid;
(3R)-6-Amino-7-hexyl-5-oxo-8-(3-trifluoromethyl-phenyl)-2,3-dihydro-5H-thiazolo[3,2-a]pyridine-3-carboxylic acid;
(3R)-7-Butyl-6-nitro-5-oxo-8-(3-trifluoromethyl-phenyl)-2,3-dihydro-5H-thiazolo[3,2-a]pyridine-3-carboxylic acid;
(3R)-6-Amino-7-(3-methyl-butyl)-5-oxo-8-(3-trifluoromethyl-phenyl)-2,3-dihydro-5H-thiazolo[3,2-a]pyridine-3-carboxylic acid;
(3R)-8-(3,4-Difluoro-phenyl)-7-heptyl-6-nitro-5-oxo-2,3-dihydro-5H-thiazolo[3,2-a]pyridine-3-carboxylic acid;
(3R)-7-octyl-5-oxo-8-[3-(trifluoromethyl)phenyl]-2,3-dihydro-5H-[1,3]thiazolo[3,2-a]pyridine-3-carboxylic acid;
(3R)-7-heptyl-5-oxo-8-(2-thienyl)-2,3-dihydro-5H-[1,3]thiazolo[3,2-a]pyridine-3-carboxylic acid;
(3R)-7-heptyl-8-(1H-indol-3-yl)-5-oxo-2,3-dihydro-5H-[1,3]thiazolo[3,2-a]pyridine-3-carboxylic acid;
(3R)-7-[3-(2,4-dichloro-phenoxy)-propyl]-5-oxo-8-phenyl-2,3-dihydro-5H-thiazolo[3,2-a]pyridine-3-carboxylic acid;
(2S,3R)-8-(3,4-difluorophenyl)-7-heptyl-2-methoxy-5-oxo-2,3-dihydro-5H-[1,3]thiazolo[3,2-a]pyridine-3-carboxylic acid;
8-(3,4-difluorophenyl)-7-heptyl-5-oxo-5H-[1,3]thiazolo[3,2-a]pyridine-3-carboxylic acid;
(2R,3R)-8-(3,4-difluorophenyl)-7-heptyl-5-oxo-2-phenyl-2,3-dihydro-5H-[1,3]thiazolo[3,2-a]pyridine-3-carboxylic acid; and
(2R,3R)-8-(3,4-difluorophenyl)-7-heptyl-2-methyl-5-oxo-2,3-dihydro-5H-[1,3]thiazolo[3,2-a]pyridine-3-carboxylic acid.

11. A process for the preparation of a compound according to claim 1 comprising reacting a compound of formula (I) with Raney® nickel to give a compound of formula (III): 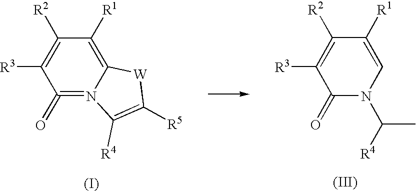
wherein R¹, R², R³, R⁴, R⁵, and W are as defined in claim 1.

12. A pharmaceutical formulation comprising a compound according to claim in admixture with a pharmaceutically acceptable adjuvant, diluent, and/or carrier.

13. A method for treating a disorder selected from thrombosis, coronary heart disease, renal fibrosis, atherosclerotic plaque formation, pulmonary disease, myocardial ischemia, atrial fibrillation, a coagulation syndrome, a thromboembolic complication of surgery, peripheral arterial occlusion, pulmonary fibrosis, cancer, polycystic ovary syndrome, diabetes, and obesity, by administering a compound according to claim 1 to a mammal.

14. The method according to claim 13 wherein the compound is combined and/or coadministered with another antithrombotic agent.
